(19) United States
(12) Patent Application Publication
(10) Pub. No.: US 2018/0327778 A1
(43) Pub. Date: Nov. 15, 2018

(54) ANIMAL MODELS FOR BRAIN INFLAMMATION AND WHITE MATTER DEGENERATION

(71) Applicant: The Regents of the University of California, Oakland, CA (US)

(72) Inventor: Xianjin Zhou, La Jolla, CA (US)

(21) Appl. No.: 15/975,500

(22) Filed: May 9, 2018

Related U.S. Application Data
(60) Provisional application No. 62/503,456, filed on May 9, 2017.

(51) Int. Cl. C12N 15/85; C12N 15/861; A01K 67/027

(52) U.S. Cl. CPC C12N 15/8509; C12N 15/8616; C12N 5/0619; C12N 2310/122; A01K 2267/0312; A01K 67/0276

(57) ABSTRACT

Provided are methods of making and using animal models for brain inflammation and white matter degeneration.

### CLAIM OF PRIORITY

This application claims the benefit of U.S. Provisional Patent Application Ser. No. 62/503,456, filed on May 9, 2017. The entire contents of the foregoing are hereby incorporated by reference.

### FEDERALLY SPONSORED RESEARCH OR DEVELOPMENT

This invention was made with Government support under Grant No. MH081037 awarded by the National Institutes of Mental Health. The Government has certain rights in the invention.

### TECHNICAL FIELD

Methods of making and using an animal model for brain inflammation and white matter degeneration are provided.

### BACKGROUND

Human multiple sclerosis (MS) is characterized with white matter degeneration and axonal injury. Experimental autoimmune/allergic encephalomyelitis (EAE) is the most extensively studied animal model for human MS. However, EAE mainly affects spinal cord white matter, whereas human MS displays demyelination and axonal injuries in the cerebral and cerebellar cortex (Pachner A R: Experimental models of multiple sclerosis. Curr Opin Neurol 2011, 24:291-299; Procaccini C, De Rosa V, Pucino V, Formisano L, Matarese G: Animal models of Multiple Sclerosis. Eur J Pharmacol 2015, 759:182-19). In addition, human MS progression cannot be studied in EAE. The other major animal model is TMEV (Theiler's murine encephalomyelitis virus) RNA virus-induced demyelination, which is considered as a more relevant model to human MS. However, demyelination is caused by persistent TMEV virus infection that is not observed in human MS (Pachner A R: Experimental models of multiple sclerosis. Curr Opin Neurol 2011, 24:291-299; Procaccini C, De Rosa V, Pucino V, Formisano L, Matarese G: Animal models of Multiple Sclerosis. Eur J Pharmacol 2015, 759:182-19). Additionally, the TMEV virus can only infect mouse, but not other rodents or primates, limiting its utilization in establishing MS models in other species. Because the TMEV virus is a mouse pathogen, animal facilities often refuse such studies (Pachner A R: Experimental models of multiple sclerosis. Curr Opin Neurol 2011, 24:291-299; Procaccini C, De Rosa V, Pucino V, Formisano L, Matarese G: Animal models of Multiple Sclerosis. Eur J Pharmacol 2015, 759:182-19).

### SUMMARY

Without wishing to be bound by theory, the present results provide evidence that scAAV8-D1shRNA-induced neuroinflammation and white matter degeneration may overcome the limitations of well-established animal models: 1) shRNA is simple, safe, efficient, and can be delivered to any brain region. 2) shRNA induces neuroinflammation and white matter degeneration likely by mimicking RNA virus infection, but without actual virus pathogens. 3) AAV episomal DNA is very stable in mouse brain and maintains expression more than 9 months post-injection [29]. We observed neuroinflammation and white matter degeneration in mice several months post-injection. 4) AAV virus can transduce many different species from rodents to primates, which enables to generate more relevant human MS models in primates. In conclusion, our studies found that massive production of double-stranded shRNAs activates immune system to cause white matter degeneration and axonal injuries that may be related to the pathogenesis of MS and other neuroinflammatory diseases. Such massive dsRNAs production can be induced in host cells by either virus infection or aberrant epigenetic alterations of host cell genome. Recent studies of tumor cells demonstrated that massive dsRNA can be produced by transcription of repetitive genomic sequences or endogenous retroviral sequences of the genome due to aberrant epigenetic modifications, which subsequently activates innate immune system to over-express interferons and pro-inflammatory cytokines [30-32]. Regardless of whether human MS is caused by virus infection or aberrant epigenetic modifications in glial and/or neuronal cells, over-production of dsRNAs are their common pathway to activate innate immune system to trigger neuroinflammation, which can be mimicked by our shRNA over-expression model.

Provided herein are methods of producing an animal model for a neuroinflammatory disease that include: introducing into the striatum of an animal a composition comprising an inhibitory nucleic acid.

Also provided herein are methods of producing an animal model for a neuroinflammatory disease that include: introducing into the hippocampus of an animal a composition comprising an inhibitory nucleic acid.

Also provided herein are methods of inducing neuroinflammation, microglial activation, white matter degeneration and injuries of large caliber axons in a rodent that include: introducing into the striatum of a rodent a composition comprising an inhibitory nucleic acid.

In some embodiments of any of the methods described herein, the inhibitory nucleic acid is further comprised within an adeno-associated virus (AAV) vector.

In some embodiments, the AAV vector is a recombinant self-complementary adeno-associated virus 8 (scAAV8) vector.

In some embodiments of any of the methods described herein, the inhibitory nucleic acid is a short hairpin RNA (shRNA).

In some embodiments, the shRNA is about 20 to 30 base pairs in length.

In some embodiments, the shRNA comprises a nucleotide sequence that is at least 80% identical to a nucleotide sequence of a dopamine D1 receptor (Drd1) gene. In some embodiments, the shRNA comprises a nucleotide sequence that is at least 80% identical to SEQ ID NO: 1, 3 or 5.

In some embodiments, the shRNA comprises a nucleotide sequence that is at least 80% identical to a nucleotide sequence of SEQ ID NO: 7.

In some embodiments, the shRNA comprises a nucleotide sequence that is at least 80% identical to a nucleotide sequence of SEQ ID NO: 13.

In some embodiments, the composition is introduced into a neuronal cell and/or a glial cell.

In some embodiments, the glial cell is a microglial cell, an astrocyte, and/or an oligodendrocyte.

In some embodiments of any of the methods described herein, the composition is introduced by stereotaxic delivery.

In some embodiments, introducing results in over-proliferation and hypertrophy of microglial cells in the striatum.

In some embodiments, the neuroinflammatory disease is selected from the group consisting of: traumatic brain injury, progressive multifocal leukoencephalopathy, Parkinson's disease and multiple sclerosis. In some embodiments, the neuroinflammatory disease is multiple sclerosis. In some embodiments, the neuroinflammatory disease is Alzheimer's disease.

In some embodiments of any of the methods described herein, the animal is a rodent.

Also provided herein are non-human transgenic animals that are models of a neuroinflammatory disease, wherein the non-human transgenic animal overexpresses a short hairpin RNA in the brain.

In some embodiments of any of the transgenic animals described herein, the short hairpin RNA comprises a nucleotide sequence that is at least 80% identical to a nucleotide sequence of a dopamine D1 receptor (Drd1) gene.

In some embodiments, the short hairpin RNA comprises a nucleotide sequence that is at least 80% identical to SEQ ID NO: 1, 3 or 5.

In some embodiments, the short hairpin RNA comprises a nucleotide sequence that is at least 80% identical to SEQ ID NO: 12.

In some embodiments, the transgenic animal is a rodent.

In some embodiments, the neuroinflammatory disease is selected from the group consisting of: traumatic brain injury, Alzheimer's disease, Parkinson's disease, progressive multifocal leukoencephalopathy, and multiple sclerosis.

In some embodiments, the short hairpin RNA was introduced into the transgenic animal by injection into the striatum of the transgenic animal.

In some embodiments, the short hairpin RNA was introduced into the transgenic animal by injection into the hippocampus of the transgenic animal.

In some embodiments, the short hairpin RNA is comprised within a viral vector.

In some embodiments, the viral vector is an associated-adenoviral (AAV) vector.

In some embodiments, viral vector is a lentivirus.

Provided herein are methods of treating a neuroinflammatory disease that include: administering a therapeutic agent to a non-human transgenic animal overexpressing a short hairpin RNA in the brain, wherein the non-human transgenic animal has a neuroinflammatory disease, to thereby treat the neuroinflammatory disease.

In some embodiments, the neuorinflammatory disease is selected from the group consisting of: traumatic brain injury, Alzheimer's disease, Parkinson's disease, progressive multifocal leukoencephalopathy, and multiple sclerosis.

In some embodiments, the short hairpin RNA comprises a nucleotide sequence that is at least 80% identical to SEQ ID NO: 1, 3 or 5.

In some embodiments, the short hairpin RNA comprises a nucleotide sequence that is at least 80% identical to SEQ ID NO: 13.

In some embodiments of any of the methods described herein, treating results in delaying disease progression, reducing neuroinflammation, reducing white matter degeneration, reducing neurodegeneration, or any combination thereof.

Provided herein are methods of determining the efficacy of a therapeutic agent in a non-human transgenic animal that include: determining a first level of neurofilament light chain (NF-L), myelin basic proteins (MBP), or both, in a first sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of NF-L, MBP, or both, in a second sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having an increased second level of NF-L, MBP, or both, as compared to the first level of NF-L, MBP, or both.

In some embodiments of any of the methods described herein, the method further includes determining a first level of ionized calcium-binding adaptor molecule 1 (Iba-1) in the first sample, determining a second level of Iba-1 in the second sample, and identifying the therapeutic agent as being effective in a non-human transgenic animal having a decreased second level of Iba-1 as compared to the first level of Iba-1.

Provided herein are methods of determining the efficacy of a therapeutic agent in a non-human transgenic animal that include: determining a first level of ionized calcium-binding adaptor molecule 1 (Iba-1) in a sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of Iba-1 in a sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having a decreased second level of Iba-1 as compared to the first level of Iba-1.

In some embodiments of any of the methods described herein, the method further includes determining a first level of NF-L, MBP, or both, in the first sample, determining a second level of NF-L, MBP, or both, in the second sample, and identifying the therapeutic agent as being effective in a non-human transgenic animal having an increased second level of NF-L, MBP, or both, as compared to the first level of NF-L, MBP, or both.

Provided herein are methods of determining the efficacy of a therapeutic agent in delaying neuroinflammatory disease progression that include: determining a first level of ionized calcium-binding adaptor molecule 1 (Iba-1) in a sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of Iba-1 in a sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having a second level of Iba-1 that is not substantially increased as compared to the first level of Iba-1.

In some embodiments of any of the methods described herein, the method further includes determining a first level of NF-L, MBP, or both, in the first sample, determining a second level of NF-L, MBP, or both, in the second sample, and identifying the therapeutic agent as being effective in a non-human transgenic animal having a second level of NF-L, MBP, or both, that is not substantially decreased as compared to the first level of NF-L, MBP, or both.

In some embodiments of any of the methods described herein, the method further includes administering one or more additional doses of the therapeutic agent identified as being effective to the non-human transgenic animal.

In some embodiments of any of the methods described herein, the neuorinflammatory disease is selected from the group consisting of: traumatic brain injury, Alzheimer's disease, Parkinson's disease, progressive multifocal leukoencephalopathy, and multiple sclerosis.

In some embodiments of any of the methods described herein, wherein the determining step includes using a method selected from the group consisting of: immunohistochemistry, immunofluorescence, an array-based method, Western blotting, and combinations thereof.

The terms “treat”, “treating” and “treatment” refer to a method of alleviating or abrogating a neuroinflammatory disease and/or its attendant symptoms. In another embodiments, treating refers to impeding or halting progression of a neuroinflammatory disease. In yet another embodiment, treating refers to extending the life of a subject with a disease. In some embodiments, treatment can result in a reduction (e.g., an about 1% to about 99% reduction, an about 1% to about 95% reduction, an about 1% to about 90% reduction, an about 1% to about 85% reduction, an about 1% to about 80% reduction, an about 1% to about 75% reduction, an about 1% to about 70% reduction, an about 1% to about 65% reduction, an about 1% to about 60% reduction, an about 1% to about 55% reduction, an about 1% to about 50% reduction, an about 1% to about 45% reduction, an about 1% to about 40% reduction, an about 1% to about 35% reduction, an about 1% to about 30% reduction, an about 1% to about 25% reduction, an about 1% to about 20% reduction, an about 1% to about 15% reduction, an about 1% to about 10% reduction, an about 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 99% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, or an about 15% to about 20% reduction) in the number, severity, and/or duration of one or more (e.g., two, three, four, five or six) symptoms and/or metrics (e.g., scores) of a neuroinflammatory disease (e.g., any of the symptoms and/or metrics of any of the neuroinflammatory diseases described herein or known in the art).

The term “therapeutically effective amount” refers to the amount of a therapeutic agent administered to a subject sufficient to treat a disease. In one embodiment, the therapeutically effective amount is sufficient to prevent development of or alleviate to some extent one or more of the symptoms of the condition or disorder being treated.

The term “subject” is defined herein to include animals, such as mammals, including but not limited to, primates (e.g., humans), cows, sheep, goats, horses, dogs, cats, rabbits, rats, mice, and the like.

As used herein, the term “biological sample” or “sample” refers to a sample obtained or derived from a subject. By way of example, the sample can include a tissue biopsy, cerebrospinal fluid, blood, serum, or plasma.

The term “population” when used before a noun means two or more of the specific noun. For example, the phrase a “population of healthy subjects” means two or more healthy subjects.

As used herein, “obtain” or “obtaining” can be any means whereby one comes into possession of the sample by “direct” or “indirect” means. Directly obtaining a sample means performing a process (e.g., performing a physical method such as extraction or tissue biopsy) to obtain a sample from the subject. Indirectly obtaining a sample refers to receiving the sample from another party or source (e.g., a third-party laboratory that directly acquired the sample). Thus, obtain is used to mean collection and/or removal of the sample from the subject. Some embodiments of any of the methods described herein can include obtaining a sample (e.g., a tissue biopsy) or samples from a subject.

The phrase “an elevated” or “an increased level” can be an elevation or an increase of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, at least 110%, at least 115%, at least 120%, at least 140%, at least 150%, at least 175%, at least 200%, at least 250%, at least 300%, at least 350%, at least 400%, at least 450%, at least 500%, or between 1% and 500%, between 1% and 450%, between 1% and 400%, between 1% and 350%, between 1% and 300%, between 1% and 250%, between 1% and 200%, between 1% and 150%, between 1% and 100%, between 1% and 50%, between 1% and 25%, between 1% and 10%, between 10% and 500%, between 10% and 400%, between 10% and 300%, between 10% and 200%, between 10% and 100%, between 10% and 50%, between 50% and 500%, between 50% and 400%, between 50% and 300%, between 50% and 200%, between 50% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, 100%, 105%, 110%, 115%, 120%, 125%, 130%, 140%, 150%, 160%, 170%, 180%, 190%, 200%, 250%, 300%, 350%, 400%, 450%, or 500%), e.g., as compared to a reference level (e.g., any of the exemplary reference levels described herein).

The phrase “a decrease”, “a reduced”, “a decreased level” or “a reduced level” can be a reduction or a decrease of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or between 1% and 100%, between 1% and 50%, between 1% and 25%, between 1% and 10%, between 1% and 5%, between 10% and 100%, between 10% and 50%, between 10% and 40%, between 10% and 30%, between 10% and 25%, between 10% and 20%, between 15% and 100%, between 15% and 75%, between 15% and 50%, between 15% and 40%, between 15% and 30%, between 15% and 20%, between 20% and 100%, between 20% and 75%, between 20% and 50%, between 20% and 40%, between 20% and 30%, between 25% and 100%, between 25% and 75%, between 25% and 50%, between 25% and 30%, between 40% and 100%, between 40% and 75%, between 40% and 50%, between 50% and 100%, between 50% and 75%, between 75% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, or 100%) e.g., as compared to a reference level (e.g., any of the exemplary reference levels described herein).

The phrase “not substantially increased” refers to clinically insignificant changes (e.g., an increase) in the second level of a particular substance or particular substances (e.g., level of Iba-1). In some embodiments, a not substantially increased second level of a particular substance or particular substances (e.g., level of Iba-1) is an increase in the second level of less than about 10% (e.g., less than about 9%, less than about 8%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%, less than about 0.005%, less than about 0.001%) as compared to the first level of the particular substance or particular substances (e.g., level of Iba-1).

In some embodiments, a not substantially increased second level of a particular substance or particular substances (e.g., level of Iba-1) is an increase in the second level of between about 0.001% and about 10% (e.g., between about 0.001% and about 5%, between about 0.001% between about 0.001% and about 2%, between about 0.001% and about 1%, between about 0.001% and about 0.5%, between about 0.001% and about 0.25%, between about 0.001% and about 0.2%, between about 0.001% and about 0.1%, between about 0.001% and about 0.05%, between about 0.001% and about 0.01%, between about 0.001% and about 0.005%, between about 0.01% and about 10%, between about 0.01% and about 5%, between about 0.01% and about 2%, between about 0.01% and about 1%, between about 0.01% and about 0.5%, between about 1% and about 10%, between about 1% and about 5%, between about 1% and about 2%, between about 2% and about 10%, between about 2% and about 6%, between about 2% and about 4%, between about 4% and about 10%, between about 4% and about 5%, between about 5% and about 10%, between about 6% and about 8%, between about 8% and 10%) as compared to the first level of the particular substance or particular substances (e.g., level of Iba-1).

The phrase “not substantially decreased” refers to clinically insignificant changes (e.g., a reduction, a decrease) in the second level of a particular substance or particular substances (e.g., level of NF-L, level of MBP). In some embodiments, a not substantially decreased second level of a particular substance or particular substances (e.g., level of NF-L, level of MBP) is a reduction or decrease in the second level of less than about 10% (e.g., less than about 9%, less than about 8%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%, less than about 0.005%, less than about 0.001%) as compared to the first level of the particular substance or particular substances (e.g., level of Iba-1).

In some embodiments, a not substantially decreased second level of a particular substance or particular substances (e.g., level of NF-L, level of MBP) is a reduction or decrease in the second level of between about 0.001% and about 10% (e.g., between about 0.001% and about 5%, between about 0.001% between about 0.001% and about 2%, between about 0.001% and about 1%, between about 0.001% and about 0.5%, between about 0.001% and about 0.25%, between about 0.001% and about 0.2%, between about 0.001% and about 0.1%, between about 0.001% and about 0.05%, between about 0.001% and about 0.01%, between about 0.001% and about 0.005%, between about 0.01% and about 10%, between about 0.01% and about 5%, between about 0.01% and about 2%, between about 0.01% and about 1%, between about 0.01% and about 0.5%, between about 1% and about 10%, between about 1% and about 5%, between about 1% and about 2%, between about 2% and about 10%, between about 2% and about 6%, between about 2% and about 4%, between about 4% and about 10%, between about 4% and about 5%, between about 5% and about 10%, between about 6% and about 8%, between about 8% and 10%) as compared to the first level of the particular substance or particular substances (e.g., level of NF-L, level of MBP).

As used herein, a “first time point” can refer, e.g., to an initial time point wherein the subject has not yet received a dose of a therapeutic agent (e.g., any of the therapeutic agents described herein). In some examples, a first time point can be, e.g., a time point when a subject has been diagnosed with a neuroinflammatory disease prior to receiving any treatment (e.g., any of the exemplary treatments described herein). In other examples, a first time point can be a time point when a subject has developed at least one (e.g., 1, 2, 3, 4, 5, 6, 7, 8, 9, or 10) symptom(s) associated with a neuroinflammatory disease (e.g., any of the exemplary symptoms associated with a neuroinflammatory disease described herein or known in the art). In some embodiments, a first time point can represent a time point after which a subject has previously received a different therapeutic agent, and the different therapeutic agent was deemed not to be successful.

As used herein, a “second time point” can refer, e.g., to a second time point after the first time point. In some examples, a subject can receive or has received at least one (e.g., 1, 2, 3, 4, 5, 6, 7, 8, 9, or 10) dose of a therapeutic agent (e.g., any of the therapeutic agents described herein) between the first and the second time points. In some embodiments, the time difference between a first and a second time point can be, e.g., 1 day, 2 days, 3 days, 4 days, 5 days, 6 days, 7 days, 8 days, 9 days, 10 days, 12 days, 14 days, 15 days, 16 days, 18 days, 20 days, 21 days, 24 days, 26 days, 28 days, 30 days, 32 days, 34 days, 36 days, 38 days, 40 days, 42 days, 49 days, 56 days, 63 days, 70 days, 77 days, 84 days, 91 days, 98 days, 105 days, 112 days, or 119 days. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A is an exemplary schematic representation of the mouse dopamine D1 receptor (Drd1) gene. Three shRNAs targeting different sites of mouse Drd1 mRNA were designed to suppress Drd1 expression. All three Drd1 shRNAs were under the control of mouse U6 promoter in scAAV-humanized renilla green fluorescent protein (hrGFP) vector.

FIG. 1B is an exemplary image of the coordinates for the stereotaxic injection of scAAV8 virus into mouse striatum. Transduction efficiency of scAAV8 was examined by hrGFP expression in mouse striatum four weeks after surgery.

FIG. 1C is an exemplary representation of immunohistochemical staining of striatal Drd1 protein using mouse anti-Drd1 antibody in control mice that were injected with scAAV8-hrGFP virus. The virus was injected only into the left hemisphere as indicated by the arrows.

FIG. 1D is an exemplary representation of immunohistochemical staining of striatal Drd1 protein using mouse anti-Drd1 antibody in mice injected with scAAV8-D1shRNA1 virus 7 weeks post-injection. The virus was injected only into the left hemisphere as indicated by the arrows.

FIG. 1E is an exemplary representation of immPRESS peroxidase-micropolymer conjugated horse anti-mouse IgG secondary antibody (without primary antibodies) immunostaining of the control mouse brain section from mice injected with scAAV8-hrGFP virus.

FIG. 1F is an exemplary representation of immPRESS peroxidase-micropolymer conjugated horse anti-mouse IgG secondary antibody (without primary antibodies) immunostaining of the control mouse brain section from mice injected with scAAV8-D1shRNA1 virus.

FIG. 1G is a representative Western blot analysis of Drd1 expression in the striatum of wild type and Drd1 knockout mice as well as mice injected with scAAV8-D1shRNA1 virus. A putative Drd1 band migrating between 100 to 200 kD (arrowhead) may be oligomers of Drd1 proteins that are absent in the knockout mice and reduced in the mice injected with scAAV8-D1shRNA1 virus. Mouse IgG heavy (50 kD) and light chains (25 kD) were detected only in the mice injected with scAAV8-D1shRNA1 virus.

FIG. 1H is a representative Western blot with the anti-mouse IgG secondary antibody only using the same samples as in FIG. 1G. Mouse IgG leakage was confirmed in mouse brain tissue transduced with scAAV8-D1shRNA1 virus.

FIG. 2A is a representative Western blot analysis of mouse IgG performed with the anti-mouse IgG secondary antibody only. Both anterior and posterior striatum were dissected from mice at different post-injection time points. Posterior: 1; anterior: 2. Control=scAAV8-hrGFP, Sh1=scAAV8-D1shRNA1, Sh2=scAAV8-D1shRNA2, Sh3=scAAV8-D1shRNA3. Equal amount of proteins were loaded and Nr1 protein was used as an internal control.

FIG. 2B is an exemplary graph comparing IgG signal intensities between different Western blots. IgG intensities were first normalized against the controls within each blot. After normalization, IgG levels from the controls and D1shRNAs were compared. The D1shRNA3 virus transduced striatum had a significantly higher level of IgG (Student's t-test, unequal variance, p<0.01) than in the striatum transduced with the control virus. Many mice injected with either scAAV8-D1shRNA1or scAAV8-D1shRNA2 virus also displayed excessive mouse IgG in their striatum.

FIG. 3A is an exemplary representation of immunohistochemical staining of ionized calcium-binding adaptor molecule 1 (Iba-1) protein, a marker of activated microglia, in mouse striatum transduced with scAAV8-hrGFP virus. The virus was injected into the left striatum only (arrow).

FIG. 3B is an exemplary representation of immunohistochemical staining of Iba-1 protein, a marker of activated microglia, in mouse striatum transduced with scAAV8-D1shRNA1 virus. The virus was injected into the left striatum only (arrow).

FIG. 3C is an exemplary representation of immunohistochemical staining of Iba-1 protein, a marker of activated microglia, in mouse striatum transduced with scAAV8-D1shRNA3virus. The virus was injected into the left striatum only (arrow).

FIG. 3D is an exemplary representation of immunohistochemical staining of Iba-1 protein, a marker of activated microglia, in mouse striatum transduced with scAAV8-D1-shRNA1 virus, showing activation of microglial cells, as determined by extensive cell proliferation and hypertrophy. Blood capillaries surrounded by activated microglial cells were markedly swollen. Scale bar: 100 μm.

FIG. 3E is an exemplary representation of immunohistochemical staining of Iba-1 protein, a marker of activated microglia, in mouse striatum transduced with scAAV8-GFP virus. No visible neuroinflammation or swelling of blood capillaries were observed in the left striatum transduced with the control scAAV8-hrGFP virus. Scale bar: 100 μm FIG. 3F is an exemplary representation of immunohistochemical staining of Iba-1 protein, a marker of activated microglia, in mouse striatum transduced with scAAV8-D1-shRNA1 virus, showing activation of microglial cells, as determined by extensive cell proliferation and hypertrophy. Blood capillaries surrounded by activated microglial cells were markedly swollen. Scale bar: 100 μm.

FIG. 3G is an exemplary representation of immunohistochemical staining of Iba-1 protein, a marker of activated microglia, in mouse striatum transduced with scAAV8-GFP virus. No visible neuroinflammation or swelling of blood capillaries were observed in the left striatum transduced with the control scAAV8-hrGFP virus. Scale bar: 100 μm FIG. 3H is an exemplary representation of a paraffin section of a scAAV8-D1shRNA mouse brain immunostained with anti-mouse IgG antibody only Anti-msIgG). Scale bar: 200 μm.

FIG. 3I is an exemplary representation of a paraffin section of a scAAV8-D1shRNA mouse brain immunostained with msIgG. Numerous IgG-positive cells scattered around a swollen blood capillary were putative peripheral immune cells (black arrow) infiltrated into the inflammatory brain tissue. Scale bar: 40 μm.

FIG. 3J is a table arbitrarily ranking microglial activation by immunohistochemical analysis in mouse brains 7 weeks post-injection. Extent of microglial activation was arbitrarily ranked (“+++” in (FIG. 3B) and (FIG. 3C), “+” in FIG. 8) in the brain sections of 34 mice injected with scAAV8-hrGFP, scAAV8-D1shRNA1, scAAV8-D1shRNA2, scAAV8-D1shRNA3 virus respectively.

FIG. 4A is an exemplary representation of immunohistochemical staining of MBP in a brain section of a mouse injected with scAAV8-hrGFP virus. The virus was injected only in the left striatum, and the analysis was performed 7 weeks post-injection.

FIG. 4B is an exemplary representation of immunohistochemical staining of MBP in a brain section of a mouse injected with scAAV8-D1shRNA1 virus. The virus was injected only in the left striatum, and the analysis was performed 7 weeks post-injection.

FIG. 4C is an exemplary representation of immunohistochemical staining of MBP in a brain section of a mouse injected with scAAV8-D1shRNA3 virus. The virus was injected only in the left striatum, and the analysis was performed 7 weeks post-injection.

FIG. 4D is an exemplary representation of immunohistochemical staining of MBP in a brain section of a mouse injected with scAAV8-D1shRNA1. MBP staining was reduced in the swollen and disorganized corpus callosum. Scale bar: 200 μm.

FIG. 4E is an exemplary representation of immunohistochemical staining of MBP in a brain section of a mouse injected with scAAV8-GFP. Scale bar: 200 μm.

FIG. 4F is an exemplary representation of immunohistochemical staining of MBP in a brain section of a mouse injected with scAAV8-D1-shRNA1. Reduction of MBP staining was also observed in striatal white matter tracts that were swollen and blebbed (arrowheads). Scale bar: 60 μm.

FIG. 4G is an exemplary representation of immunohistochemical staining of MBP in a brain section of a mouse injected with scAAV8-GFP. Scale bar: 60 μm.

FIG. 5A is an exemplary representation of a paraffin section immunostained with anti-Iba-1 protein in a mouse injected with scAAV8-hrGFP.

FIG. 5B is an exemplary representation of a paraffin section immunostained with anti-neurofilament light chain (NF-L) protein in a mouse inject with scAAV8-hrGFP. This paraffin section is consecutive to the section shown in FIG. 5A.

FIG. 5C is an exemplary representation of a paraffin section immunostained with anti-Iba-1 protein in a mouse injected with scAAV8-D1shRNA1. Activated microglial cells were preferentially concentrated on corpus callosum.

FIG. 5D is an exemplary representation of a paraffin section immunostained with anti-NF-L protein in a mouse injected with scAAV8-D1shRNA1. This paraffin section is consecutive to the section shown in FIG. 5C. Activated microglial cells were preferentially concentrated on corpus callosum. Activated microglial cells were preferentially concentrated on corpus callosum.

FIG. 5E is an exemplary representation of a paraffin section immunostained with anti-Iba-1 protein in a mouse injected with scAAV8-D1shRNA3.

FIG. 5F is an exemplary representation of a paraffin section immunostained with anti-NF-L protein in a mouse injected with scAAV8-D1shRNA3. This paraffin section is consecutive to the section shown in FIG. 5E.

FIG. 5G is an exemplary representation of a high magnification of immunostaining of NF-L in mice injected with scAAV8-D1shRNA1. Neurofilaments were reduced in the swollen and disorganized corpus callosum. Scale bar: 200 μm.

FIG. 5H is an exemplary representation of a high magnification of immunostaining of anti-NF-L in mice injected with scAAV8-GFP. Scale bar: 200 μm.

FIG. 5I is an exemplary representation of immunostaining of anti-NF-L in mice injected with scAAV8-D1shRNA1. Striatal white matter tracts were also swollen and blebbed (arrowhead) in the left striatum transduced with scAAV8-D1shRNA1.Scale bar: 60 μm.

FIG. 5J is an exemplary representation of immunostaining of anti-NF-L in mice injected with scAAV8-GFP. Scale bar: 60 μm.

FIG. 6A is an exemplary representation of paraffin sections from mice injected with scAAV8-D1shRNA3 virus immunostained with anti-Iba-1. A high density of activated microglial cells (anti-Iba-1 staining) was shown in the left striatum injected with scAAV8-D1shRNA3 virus. Scale bar: 120 μm.

FIG. 6B is an exemplary representation of paraffin sections from mice injected with scAAV8-GFP virus immunostained with anti-Iba-1. Scale bar: 120 μm.

FIG. 6C is an exemplary representation of paraffin sections from mice injected with scAAV8-GFP virus immunostained with anti-NF-L. This section is adjacent to the section of FIG. 6A. Decreased overall NF-L staining and various NF-L aggregates were observed in the left side corpus callosum transduced with scAAV8-D1shRNA3 virus Scale bar: 120 μm.

FIG. 6D is an exemplary representation of paraffin sections from mice injected with scAAV8-GFP virus immunostained with anti-NF-L. This section is adjacent to the section of FIG. 6A.

FIG. 6E is an exemplary representation of a high magnification of the region highlighted in FIG. 6C. Scale bar: 50 μm.

FIG. 6F is an exemplary representation of a high magnification of the region highlighted in FIG. 6D. Scale bar: 50 μm.

FIG. 7A shows three different shRNAs that were designed to target different sites of mouse Drd1 gene: D1shRNA1: 5′-AAT ACC CTT GTC TGT GCC GCT-Loop-AGC GGC ACA GAC AAG GGT ATT tttttt-3′ (SEQ ID NO: 1); 3′-TTA TGG GAA CAG ACA CGG CGA-Loop-TCG CCG TGT CTG TTC CCA TAA aaaaaa-5′ (SEQ ID NO: 2); D1shRNA2: 5′-GGC CCT TGG ATG GCA ATT TTA CT-Loop-AG TAA AAT TGC CAT CCA AGG GCC ttttttt-3′ (SEQ ID NO:3); 3′-CCG GGA ACC TAC CGT TAA AAT GA-Loop-TC ATT TAA ACG GTA GGT TCC CGG aaaaaaa-5′ (SEQ ID NO: 4); D1shRNA3: 5′-CCC TAG GGC TTG CTG TTA AGA AA-Loop-TT TCT TAA CAG CAA GCC CTA GGG ttttttt-3′ (SEQ ID NO: 5); 3′-ggg atc ccg aac gac aat tct tt-Loop-AA AGA ATT GTC GTT CGG GAT CCC aaaaaaa-5′ (SEQ ID NO: 6).

FIG. 7B is an exemplary representation of an immunohistochemical staining of Drd1 using rabbit anti-Drd1 antibody in mouse striatum transduced with scAAV8-hrGFP virus. The virus was injected into the left striatum only.

FIG. 7C is an exemplary representation of an immunohistochemical staining of Drd1 using rabbit anti-Drd1 antibody in mouse striatum transduced with scAAV8-D1shRNA1 virus. The virus was injected into the left striatum only. Reduced Drd1 protein was observed in the left striatum.

FIG. 7D is an exemplary representation of a high magnification of the region highlighted of FIG. 7B. Scale bar: 50 μm.

FIG. 7E is an exemplary representation of a high magnification of the region highlighted of FIG. 7C. Scale bar: 50 μm.

FIG. 8A is an exemplary representation of an immunohistochemical staining of Iba-1 in mice injected with scAAV8-D1shRNA2 virus.

FIG. 8B is an exemplary representation of a high magnification of a region highlighted in FIG. 8A. Microglial cells were activated to surround blood capillaries in the left striatum transduced with the virus, indicating mild neuroinflammation around the blood capillaries. Scale bar: 50 μm.

FIG. 8C is an exemplary representation of a high magnification of a region highlighted in FIG. 8A. Microglial cells were activated to surround blood capillaries in the left striatum transduced with the virus, indicating mild neuroinflammation around the blood capillaries. Scale bar: 50 μm.

FIG. 9A is an exemplary representation of an immunohistochemical staining of MBP in the left corpus callosum transduced with scAAV8-hrGFP virus Scale bar: 100 μm.

FIG. 9B is an exemplary representation of an immunohistochemical staining of MBP in the right corpus callosum without virus injection of the same mouse as in FIG. 9A. Scale bar: 100 μm.

FIG. 9C is an exemplary representation of an immunohistochemical staining of MBP in the left corpus callosum transduced with scAAV8-D1shRNA3 virus. MBP was reduced (black arrow) in the left side corpus callosum Scale bar: 200 μm.

FIG. 9D is an exemplary representation of an immunohistochemical staining of MBP in the right corpus callosum without virus injection of the same mouse as in FIG. 9C. Scale bar: 100 μm.

FIG. 9E is an exemplary representation of a high magnification of a region highlighted in FIG. 9C. MBP staining was decreased in striatal white matter tracts that were swollen and blebbed (black arrowheads). Scale bar: 60 μm.

FIG. 9F is an exemplary representation of a high magnification of a region highlighted in FIG. 9D. Scale bar: 60 μm.

FIG. 10A is an exemplary representation of an immunohistochemical staining of anti-Iba-1 antibody in the left striatum transduced with scAAV8-hrGFP virus 7 weeks post-injection. There is no activation of microglial cells in the left striatum transduced with scAAV8-hrGFP virus.

FIG. 10B is an exemplary representation of an immunohistochemical staining of anti-NF-L antibody in the left striatum transduced with scAAV8-hrGFP virus 7 weeks post-injection. This section is a consecutive paraffin section of the section of FIG. 10A.

FIG. 10C is an exemplary representation of a high magnification of a region highlighted in the left side of the corpus callosum of FIG. 10B. There is no reduction of NF-L staining in the corpus callosum on the left side. Scale bar: 150 μm.

FIG. 10D is an exemplary representation of a high magnification of a region highlighted in the right side of the corpus callosum of FIG. 10 B. Scale bar: 150 μm.

FIG. 11A is an exemplary representation of an immunohistochemical staining of anti-Iba-1 in the striatum transduced with scAAV8-D1shRNA3 virus, showing white matter degeneration and neurofilament reduction. Extensive microglial activation as shown by anti-Iba-1 immunostaining was observed in the left striatum transduced with the virus in comparison with the control right striatum.

FIG. 11B is an exemplary representation of an immunohistochemical staining of anti-NF-L in the striatum transduced with scAAV8-D1shRNA3 virus.

FIG. 11C is an exemplary representation of a high magnification of a region highlighted in the left side corpus callosum of FIG. 11B. Decreased NF-L staining was observed in the left side corpus callosum. Scale bar: 200 μm.

FIG. 11D is an exemplary representation of a high magnification of a region highlighted in the control right side corpus callosum of FIG. 11B. Scale bar: 200 μm.

FIG. 11E is an exemplary representation of a high magnification of a region highlighted in the left side corpus callosum of FIG. 11C. Neurofilament staining was reduced in blebbed striatal white matter tracts (black arrow).Scale bar: 60 μm.

FIG. 11F is an exemplary representation of a high magnification of a region highlighted in the control right side corpus callosum of FIG. 11D.

FIG. 12A is an IHC image showing mouse anti-IgG staining in a mouse seven weeks after injection with scAAV8-hrGFP (control) into the left hippocampal dentate gyrus as shown by arrow in inset. Red arrowheads point to blood capillaries between DG and thalamus (TH). Scale bar: 40 μm.

FIG. 12B is an IHC image showing anti-Iba-1 staining in a mouse seven weeks after injection with scAAV8-hrGFP (control) into the left hippocampal dentate gyrus as shown by arrow in inset. Scale bar: 40 μm.

FIG. 12C is an IHC image showing mouse anti-IgG staining in a mouse seven weeks after injection with scAAV8-D1shRNA1 into the left hippocampal dentate gyrus as shown by arrow in inset. Red arrowheads point to blood capillaries between DG and TH. Scale bar: 40 μm.

FIG. 12D is an IHC image showing anti-Iba-1 staining in a mouse seven weeks after injection with scAAV8-D1shRNA1 into the left hippocampal dentate gyrus. Scale bar: 40 μm.

FIG. 13A shows an IHC image of coronal brain sections from the scAAV8-D1shRNA mice immunostained with anti-mouse IgG antibodies, and co-stained with hematoxylin for nuclei. The left dentate gyrus was injected with the scAAV8-D1shRNA1 virus as shown by arrows in insets.

FIG. 13 B shows an IHC image of the right dentate gyrus receiving no virus injection.

FIG. 13C shows an IHC image of immunostaining of GluR1 proteins. GluR1 proteins were completely wiped out in the degenerating left dentate gyrus.

FIG. 13D shows an IHC image of immunostaining of GluR1 proteins in the control right dentate gyrus of the same brain section as in FIG. 13C.

FIG. 14 shows IHC images showing neuroinflammation and white matter degeneration in striatum of scAAV8-shRNA mice 7 weeks after scAAV8-hrGFP (control) and scAAV8-shRNA virus injection into the left striatum. Anti-msIgG was used to detect IgG extravasation in striatum. Anti-Iba-1 was used to detect microglial activation.

FIG. 15A is an IHC image showing microglial activation by anti-Iba-1 staining 7 weeks after scAAV8-shRNA virus injection of the left striatum.

FIG. 15B is an IHC image showing mild neuroinflammation by anti-mouse IgG antibody. Infiltration of IgG-positive peripheral immune cells (red arrowheads) was observed in the inflammatory striatum.

### DETAILED DESCRIPTION

RNA interference (RNAi) is a host defense system evolved to destroy invading virus. Viral dsRNA is recognized and cut by Dicer into short interfering RNAs that are loaded into RISC (RNA-induced silencing complex) to specifically cleave viral RNAs [25]. During this process, innate immune response is activated to increase expression of interferons and inflammatory cytokines to combat the invading virus [6-8]. shRNA is processed by the same Dicer-RISC pathway to mediate gene silencing effects. As shown herein, sustained overexpression of D1shRNAs driven by U6 promoter may have activated innate immune responses in mouse striatum transduced by scAAV8-D1shRNA virus and thereby initiated neuroinflammation. It is possible that transduction of oligodendrocytes may directly contribute to white matter degeneration and axonal injuries. Recent studies suggest that shRNA over-expression can disturb endogenous microRNA biogenesis [26]. Given that microRNA and RNAi share the same molecular biogenesis pathway, over-expression of shRNAs may disturb both RNAi-mediated defense system and innate immune system to induce neuroinflammation.

Without wishing to be bound by theory and as a proof of concept, the present inventor has shown that mice transduced with recombinant scAAV8 virus to express dopamine D1 receptor short-hairpin RNA (D1shRNA) or a random shRNA develop white matter degeneration and neuroinflammation. To silence expression of striatal dopamine D1 receptor (Drd1), mouse striatum was transduced with recombinant scAAV8 virus (self-complementary AAV8) to express Drd1 short-hairpin RNAs (shRNA). Striatal Drd1 protein was reduced by D1shRNA expression. Surprisingly, transduction of mouse striatum with scAAV8-D1shRNA virus, but not the control scAAV8-hrGFP virus, causes extensive microglial activation, markedly swelling of blood capillaries, massive leakage of mouse IgG, white matter degeneration, and axonal injuries. RNA interference is known to be coupled to innate immune system in host cell defense against virus infection. Without wishing to be bound by theory, the present results demonstrate that over-production of shRNAs triggers immune responses in brain. Transduction with scAAV8-D1shRNA virus may therefore provide a simple method to induce extensive neuroinflammation and white matter degeneration that are related to multiple sclerosis and other neuroinflammatory diseases.

### Methods of Producing an Animal Model

The present methods include the use of one or more of a inhibitory nucleic acid (e.g., a random shRNA sequence, a D1shRNA) for producing an animal model for a neuroinflammatory disease (e.g., multiple sclerosis, Alzheimer's disease, Parkinson's disease, progressive multifocal leukoencephalopathy) that include introducing into the striatum of an animal a composition comprising an inhibitory nucleic acid (e.g., an inhibitory nucleic acid that comprises a random nucleic acid sequence of between 19 to 30 nucleotides, an inhibitory nucleic acid that is at least 80% identical to a nucleotide sequence of dopamine D1 receptor (Drd1) gene (e.g., a striatal Drd1 gene or a mouse striatal Drd1 gene)).

The term “animal” refers to any non-human mammal. In some embodiments, the animal is a rodent (e.g., a mouse, a guinea pig, a hamster or a rat).

In some embodiments, the composition comprising an inhibitory nucleic acid is introduced directly into the striatum of the animal. In some embodiments, the composition comprising an inhibitory nucleic acid is introduced directly into the hippocampus of the animal. In some embodiments, the composition is introduced using stereotaxic delivery. Methods of introducing a composition using stereotaxic delivery into the brain are known in the art. Briefly, stereotaxic delivery (also known as stereotactic surgery) is a surgical procedure in which three-dimensional coordinates are used to precisely locate an area of interest of the anatomy and deliver compositions (e.g., a composition comprising an inhibitory nucleic acid) to the identified area. See, e.g., Cetin et al. (2006) Nat. Protoc. 1(6): 3166-73; JoVE Science Education Database. Essentials of Neuroscience. Rodent Stereotaxic Surgery. JoVE, Cambridge, Mass., (2017); each incorporated in its entirety herein. Inhibitory nucleic acids have been employed in the use of producing animal models of diseases states.

The term “introduce” or “introducing” as used herein refers to the administration of a composition comprising an inhibitory nucleic acid that targets a Drd1 gene (e.g., striatal Drd1). Introducing can occur using a single or multiple administration (e.g., injections) of any composition as described herein. The dosage and frequency of introducing depends on the requirement of inhibition of Drd1 expression and/or activity and by the amount that is tolerated by the animal model. In one aspect, introducing includes stereotaxic delivery. In yet another aspect, introducing results in over-proliferation and hypertrophy of microglial cells (e.g., hypertrophy of microglial cells in the striatum).

Provided herein are non-human transgenic animals that can be used to study the development and/or progression of a neuroinflammatory disease (e.g., multiple sclerosis, Alzheimer's disease, Parkinson's disease, progressive multifocal leukoencephalopathy) or to determine the therapeutic potential of a compound to designed to treat a neuroinflammatory disease (e.g., multiple sclerosis, Alzheimer's disease, Parkinson's disease, progressive multifocal leukoencephalopathy). The non-human transgenic animals provided herein can overexpress a composition that includes an inhibitory nucleic acid that comprises a random nucleic acid sequence of between 19 to 30 nucleotides. In one aspect, the non-human transgenic animals provided herein can overexpress a composition that includes an inhibitory nucleic acid that targets (e.g., inhibits) expression of a Drd1 gene (e.g., a striatal Drd1 gene). Any of the inhibitory nucleic acids described herein may be over-expressed in the animal for more than 9 months (e.g., 9, 10, 11, 12, 13, 14, 15, 16, 18 or 20 months; see, e.g., Rahim et al. (2012) Gene Ther 19:936-946.

In some aspects of any of the non-human transgenic animals described herein, the composition that includes an inhibitory nucleic acid can also include a vector (e.g., any of the vector described herein). In some embodiments, the composition includes an adeno-associated virus (AAV), wherein the AAV includes a reporter sequence encoding a reporter protein. Non-limiting examples of reporter proteins include DNA sequences encoding: a beta-lactamase, a beta-galactosidase (LacZ), an alkaline phosphatase, a green fluorescent protein (GFP), a red fluorescent protein (RFP), an mCherry fluorescent protein, a yellow fluorescent protein, and a luciferase. Additional examples of reporter sequences are known in the art. When associated with regulatory element which drive their expression, the reporter sequence can provide signals detectable by conventional means, including enzymatic, radiographic, colorimetric, fluorescence, or other spectrographic assay; fluorescence activating cell sorting (FACS) assay; immunological assays (e.g., enzyme linked immunosorbent assay (ELISA), radioimmunoassay (RIA), immunohistochemistry, immunofluorescence, in vivo optical imaging systems (IVIS).

In some embodiments, the reporter sequence is the LacZ gene, and the presence of a vector carrying the LacZ gene in a mammalian cell (e.g., a neuronal cell) is detected by assays for beta-galactosidase activity. When the reporter is a fluorescent protein (e.g., GFP or luciferase), the presence of a vector carrying the fluorescent protein or luciferase in a mammalian cell (e.g., a neuronal cell) may be measured by fluorescent techniques (e.g., fluorescent microscopy, FACS, IVIS) or light production in a luminometer (e.g., a spectrophotometer or an IVIS imaging instrument). In some aspects where the reporter is luciferase, the promoter of a gene that is expressed during neuroinflammation (e.g., retinoic acid-inducible gene I (RIG-I)) is upstream of the luciferase gene in an expression vector.

### Neuroinflammatory Diseases

Provided herein are method of producing an animal model for the study of a neuroinflammatory disease. In some embodiments, the animal model recapitulates a human disease (e.g., a neuroinflammatory disease) in terms of site of onset, rate of progression, biochemical modifications, clinical manifestation of symptoms. In some embodiments, the animal model is a model for a neuroinflammatory disease (e.g., Alzheimer's disease, Parkinson's disease, progressive multifocal leukoencephalopathy or multiple sclerosis. In some embodiments, the animal model develops neuroinflammation, microglial activation, white matter degeneration and/or injuries of large caliber axons. In some embodiments, the neuroinflammatory disease is intermittent and/or relapsing (e.g., relapsing-remitting multiple sclerosis (RRMS)). In some embodiments, the neuroinflammatory disease is resistant to a previously administered treatment (i.e., a previously administered treatment was determined to be ineffective to treating the neuroinflammatory disease).

As used herein, the term “neuroinflammatory disease” refers to a collection of diseases/disorders wherein the brain and/or spinal cord are inflamed (e.g., multiple sclerosis, Alzheimer's disease, Parkinson's disease, progressive multifocal leukoencephalopathy). Neuroinflammatory diseases are characterized by immune cells that attack the central nervous system. In one aspect, a neuroinflammatory disease is caused by an infection, toxic metabolites, neurotoxins, and/or traumatic brain injury. A neuroinflammatory disease can be acute, chronic, low-grade, or high grade.

In some aspects, a neuroinflammatory disease is associated with white matter degeneration, and/or neurodegeneration. In some embodiments, neurodegeneration is localized to the site of injection of a composition that includes an inhibitory nucleic acid (e.g., a composition that includes a shRNA). In other embodiments, neurodegeneration can spread beyond the site of injection.

Various methods are known in the art to determine neuroinflammation, microglial activation, white matter degeneration, neurodegeneration and injuries of large caliber axons. Neuroinflammation can be measured by determining the presence and/or amount of infiltration of peripheral immune cells. In one aspect, neurodegeneration is measured by determining the presence and/or absence of neurofilament light chain (NF-L), an abundant structure protein of large-caliber axons in a brain tissue sample as compared to a reference level of NF-L in a brain tissue sample of a reference sample (e.g., any of the reference samples described herein). Non-limiting methods of determining NF-L include: immunohistochemistry, immunofluorescence, in situ RNA hybridization.

A decrease in NF-L expression and/or activity can be a reduction or a decrease in expression and/or activity of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or between 1% and 100%, between 1% and 50%, between 1% and 25%, between 1% and 10%, between 1% and 5%, between 10% and 100%, between 10% and 50%, between 10% and 40%, between 10% and 30%, between 10% and 25%, between 10% and 20%, between 15% and 100%, between 15% and 75%, between 15% and 50%, between 15% and 40%, between 15% and 30%, between 15% and 20%, between 20% and 100%, between 20% and 75%, between 20% and 50%, between 20% and 40%, between 20% and 30%, between 25% and 100%, between 25% and 75%, between 25% and 50%, between 25% and 30%, between 40% and 100%, between 40% and 75%, between 40% and 50%, between 50% and 100%, between 50% and 75%, between 75% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, or 100%) e.g., as compared to a reference level (e.g., any of the exemplary reference levels described herein).

In yet another aspect, neurodegeneration is measured by determining the presence and/or absence of Iba-1, a marker of microglial activation in a brain tissue sample as compared to a reference level of Iba-1 in a brain tissue sample of a reference sample (e.g., any of the reference samples described herein). Non-limiting methods of determining Iba-1 include: immunohistochemistry, immunofluorescence, in situ RNA hybridization.

An increase in Iba-1 expression and/or activity can be an elevation or an increase in expression and/or activity of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, at least 110%, at least 115%, at least 120%, at least 140%, at least 150%, at least 175%, at least 200%, at least 250%, at least 300%, at least 350%, at least 400%, at least 450%, at least 500%, or between 1% and 500%, between 1% and 450%, between 1% and 400%, between 1% and 350%, between 1% and 300%, between 1% and 250%, between 1% and 200%, between 1% and 150%, between 1% and 100%, between 1% and 50%, between 1% and 25%, between 1% and 10%, between 10% and 500%, between 10% and 400%, between 10% and 300%, between 10% and 200%, between 10% and 100%, between 10% and 50%, between 50% and 500%, between 50% and 400%, between 50% and 300%, between 50% and 200%, between 50% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, 100%, 105%, 110%, 115%, 120%, 125%, 130%, 140%, 150%, 160%, 170%, 180%, 190%, 200%, 250%, 300%, 350%, 400%, 450%, or 500%), e.g., as compared to a reference level (e.g., any of the exemplary reference levels described herein).

In one aspect, neurodegeneration is measured by determining the extent of vascular and/or microvascular permeability in the brain. The extent of vascular permeability includes the permeability of the blood brain barrier. Vascular permeability can be measured by determining the extent of vascular leakage, extravasation of antibody into the brain of a subject. Methods of determining vascular permeability are known in the art and are described herein. For example, vascular permeability can be measured by Evans blue dye extrusion, endothelial barrier antigen (EBA) staining, and monoclonal antibody leakage (e.g., radiolabeled monoclonal antibody leakage).

In one aspect, white matter degeneration is measured by determining the presence and/or absence of myelin basic proteins (MBP) in a brain tissue sample as compared to a reference level of MBP in a brain tissue sample of a reference sample (e.g., any of the reference samples described herein). Non-limiting methods of determining MBP include: immunohistochemistry, immunofluorescence, in situ RNA hybridization.

A decrease in MBP expression and/or activity can be a reduction or a decrease in expression and/or activity of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or between 1% and 100%, between 1% and 50%, between 1% and 25%, between 1% and 10%, between 1% and 5%, between 10% and 100%, between 10% and 50%, between 10% and 40%, between 10% and 30%, between 10% and 25%, between 10% and 20%, between 15% and 100%, between 15% and 75%, between 15% and 50%, between 15% and 40%, between 15% and 30%, between 15% and 20%, between 20% and 100%, between 20% and 75%, between 20% and 50%, between 20% and 40%, between 20% and 30%, between 25% and 100%, between 25% and 75%, between 25% and 50%, between 25% and 30%, between 40% and 100%, between 40% and 75%, between 40% and 50%, between 50% and 100%, between 50% and 75%, between 75% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, or 100%) e.g., as compared to a reference level (e.g., any of the exemplary reference levels described herein).

In yet other aspects, neurodegeneration is determined by histological analysis. For example, histological analysis can identify the loss of structures (e.g., disorganization of corpus callosum), cellular swelling, and apoptosis. Various methods are known in the art to detect degeneration and apoptotic cells (e.g., terminal deoxynucleotidyl transferase dUTP nick end labeling).

As used herein, the term “multiple sclerosis” refers to a disease of the central nervous system (i.e. a disease that affects the brain and spinal cord). Multiple sclerosis (MS) is an autoinflammatory disease in which the immune system attacks myelin. During MS, oligodendrocytes are destroyed and neuronal function is progressively lost (Compton et al. (2008) Lancet 372(9648): 1502-17). While MS is believed to be a partially heritable autoimmune disease—increased risk in developing MS if someone related has MS, environmental factors such as Epstein-Barr virus infection, and smoking are believed to contribute to MS susceptibility (Simpson et al. Mult. Scler. (2015) 21(8): 969-77). Current treatment options have been reviewed by Parnell and Booth (2017) Front Immunol. 8: 425; Staun-Ram and Miller (2017) Clin. Immunol. Pii: S1521-6616(16)30556 and Mills et al. (2017) Front Neurol. 8: 116. Human MS is characterized by white matter degeneration and axonal injury.

Dopamine D1 receptor (Drd1)

Striatal dopamine D1 receptor (Drd1) plays an important role in a number of neuroinflammatory diseases including Parkinson disease, Huntington disease, addiction, depression and schizophrenia [14-18]. Drd1 is the most abundant dopamine receptor in the central nervous system. Drd1 is a G-protein coupled receptor that activates cyclic adenosine monophosphate (AMP)-dependent protein kinases. In some embodiments, the Drd1 gene comprises a mouse Mus musculus Drd1 gene (e.g., a striatal Drd1 gene). In some embodiments, the Drd1 gene comprises the nucleotide sequence encoding the Drd1 protein (Accession Number: NM_001291801.1; SEQ ID NO: 7; or NM_010076.3; SEQ ID NO: 8).

### Inhibitory Nucleic Acids & Compositions

Inhibitory nucleic acids useful in the present methods, compositions and non-human transgenic animals include those that are designed to inhibit a gene (e.g., a striatal Drd1 gene). A nucleic acid that “specifically binds” binds primarily to the target, i.e. Drd1, but not to other non-target RNAs. The specificity of the nucleic acid interaction thus refers to its function (e.g., inhibiting Drd1) rather than its hybridization capacity.

Oligonucleotides may exhibit nonspecific binding to other sites in the genome or other mRNAs, without interfering with binding of other regulatory proteins and without causing degradation of the non-specifically-bound RNA. Thus, this nonspecific binding does not significantly affect function of other non-target RNAs. Inhibitory nucleic acids useful in the methods of producing, compositions and non-human transgenic animals described herein include inhibitory nucleic acids that decrease the expression and/or activity of a gene (e.g., a Drd1 gene). Inhibitory nucleic acids useful in the present methods, compositions and non-human transgenic animals include antisense oligonucleotides, siRNA compounds, single- or double-stranded RNA interference (RNAi) compounds and other oligonucleotide mimetics which hybridize to at least a portion of the target nucleic acid and modulate its function. In some embodiments, the inhibitory nucleic acid includes an antisense RNA, an antisense DNA, a chimeric antisense oligonucleotide, a short interfering RNA (siRNA), a short hairpin RNA (shRNA) or a combination thereof. In some embodiments, the composition comprises an inhibitory nucleic acid that is selected from the group consisting of: an antisense molecule, a small interfering RNA, a double-stranded RNA, a small hairpin RNA (shRNA). In some embodiments, the composition comprises a small RNA with triphosphate at the 5′ end.

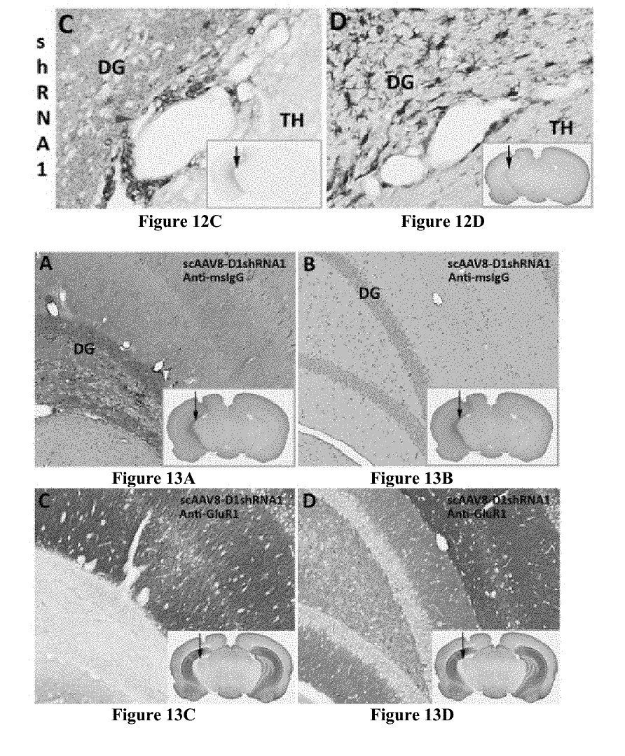

RNA interference has been widely used to suppress gene expression in mammalian cells [1] and in vivo [2-4]. As an off-target effect, both long and short interference RNAs, recognized as viral double-stranded RNAs by host cells [5], activate innate immune system to induce expression of pro-inflammatory cytokines via either sequence-independent or -dependent pathways [6-8]. Short hairpin RNA (shRNA), however, was suggested to have much less immunogenicity since it is processed by endogenous microRNA pathway [9, 10]. Different viral vectors have been developed to express shRNA in target tissues. Among these vectors, self-complementary adeno-associated virus (scAAV) with a double-stranded DNA bypasses the rate-limiting step of second DNA strand synthesis in traditional AAV virus (single-stranded DNA) and thereby transduces target tissue up to 100-fold more effectively [11-13]. AAV episomal DNA is very stable in mouse brain and maintains expression more than 9 months post-injection [29]. As shown in the Examples below and in FIG. 14, animals injected with either a random shRNA sequence or a shRNA targeting D1 with AAV developed neuroinflammation, white matter degeneration and neurodegeneration, while these effects were not seen in animals injected with AAV8 including humanized renilla green fluorescent protein (hrGFP). These findings demonstrate that neuroinflammation is not caused by AAV and that the development of neuroinflammation occurs in the absence of targeted shRNA gene silencing. Instead, shRNA immunogenicity induces neuroinflammation as shown in FIG. 14.

SEQ ID NO: 1-6 are exemplary shRNAs that target Drd1. D1shRNA1: 5′-AAT ACC CTT GTC TGT GCC GCT-Loop-AGC GGC ACA GAC AAG GGT ATT tttttt-3′ (SEQ ID NO: 1); 3′-TTA TGG GAA CAG ACA CGG CGA-Loop-TCG CCG TGT CTG TTC CCA TAA aaaaaa-5′ (SEQ ID NO: 2); D1shRNA2: 5′-GGC CCT TGG ATG GCA ATT TTA CT-Loop-AG TAA AAT TGC CAT CCA AGG GCC ttttttt-3′ (SEQ ID NO:3); 3′-CCG GGA ACC TAC CGT TAA AAT GA-Loop-TC ATT TAA ACG GTA GGT TCC CGG aaaaaaa-5′ (SEQ ID NO: 4); D1shRNA3: 5′-CCC TAG GGC TTG CTG TTA AGA AA-Loop-TT TCT TAA CAG CAA GCC CTA GGG ttttttt-3′ (SEQ ID NO: 5); 3′-ggg atc ccg aac gac aat tct tt-Loop-AA AGA ATT GTC GTT CGG GAT CCC aaaaaaa-5′ (SEQ ID NO: 6).

In some embodiments, the Drd1 inhibitory nucleic acid comprises SEQ ID NO: 1, 3 or 5. In some embodiments, the Drd1 inhibitory nucleic acid comprises SEQ ID NO:1. In some embodiments, the Drd1 inhibitory nucleic acid comprises SEQ ID NO:3. In some embodiments, the Drd1 inhibitory nucleic acid comprises SEQ ID NO:5. In some embodiments, the Drd1 inhibitory nucleic acid is a nucleic acid comprising a sequence that is complementary to a contiguous sequence of at least 5 (e.g., at least 6, at least 7, at least 8, at least 9, at least 10, at least 11, at least 12, at least, 13, at least 14, at least 15, at least 16, at least 17, at least 18, at least 19, at least 20, at least 21, 5, 6, 7, 8, 9, 10, 11, 12, 13, 14, 15, 16, 17, 18, 19, 20, 21, or 22) nucleotides present in SEQ ID NO: 7. In some embodiments, the Drd1 inhibitory nucleic acid (e.g., shRNA) is 20 to 30 (e.g., 20 to 22, 20 to 23, 20 to 25, 20 to 26, 20 to 28, 21 to 22, 21 to 23, 21 to 25, 21 to 26; 20, 21, 22, 23, 24, 25, 26, 27, 28, 29 or 30) base pairs in length.

In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 1, 2, 3, 4, 5 or 6. In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 1. In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 2. In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 3. In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 4. In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 5. In some embodiments, the Drd1 inhibitory nucleic acid (e.g., shRNA) is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 6.

The inhibitory nucleic acid described herein can be included in any of the compositions described herein for use in any of the methods and non-human transgenic animals provided herein. The compositions provided herein can include a vector that encodes an inhibitory nucleic acid that targets any gene (e.g., an inhibitory nucleic acid that targets a Drd1 gene).

In some embodiments, the inhibitory nucleic acid does not specifically target any gene. In some embodiments, the inhibitory nucleic acid comprises a scrambled or random short hairpin RNA sequence of about 19 to 30 base pairs in length (e.g., about 19 to 28 base pairs, about 19 to 26 base pairs, about 19 to 25 base pairs, about 19 to 24 base pairs, about 19 to 22 base pairs, about 19 to 20 base pairs, about 20 to 30 base pairs, about 20 to 28 base pairs, about 20 to 26 base pairs, about 20 to 25 base pairs, about 20 to 24 base pairs, about 20 to 22 base pairs, about 22 to 30 base pairs, about 22 to 28 base pairs, about 22 to 26 base pairs, about 22 to 25 base pairs, about 22 to 24 base pairs; 19 base pairs, 20 base pairs, 21 base pairs, 22 base pairs, 23 base pairs, 24 base pairs, 25 base pairs, 26 base pairs, 27 base pairs, 28 base pairs, 29 base pairs, or 30 base pairs)

In some embodiments, the random short hairpin RNA (shRNA) sequence consists of or comprises: CCTAAGGTTAAGTCGCCCTCGCTCGAGCGAGGGCGACTTAACCTTAGG (SEQ ID NO: 12). In some embodiments, the random shRNA is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93% 94%, 95%, 96%, 97%, 98%, 99% or 100%) identical to SEQ. ID NO: 12.

In some embodiments, the random short hairpin RNA (shRNA) sequence consists of or comprises: AACGTGTGAAGCTTTAACTAACTTCCTGTCATTAGTTAAAGCTTCACACGTT (SEQ ID NO: 13). In some embodiments, the random shRNA is a nucleic acid comprising a sequence that is at least 80% (e.g., at least 85%, at least 90%, at least 95%, 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 13.

In some embodiments, the vector is a viral vector. Non-limiting examples of viral vectors include: lentivirus, retrovirus, adeno-associated virus. In some embodiments, the composition includes an adeno-associated virus vector (e.g., a self-complementary AAV vector). In some embodiments, the self-complementary AAV vector is a self-complementary AAV serotype 8 (scAAV8) vector. The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168, 1990). The ITR sequences are about 145 by in length. In some embodiments, at least 75% of the sequences (e.g., at least 80%, at least 85%, at least 90%, or at least 95%) encoding the ITRs are incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the vectors described herein are flanked by 5′ and 3′ AAV ITR sequences. The AAV ITR sequences may be obtained from any known AAV, including presently identified mammalian AAV types.

Any of the vectors described herein can further include an internal terminal repeat (ITR), a promoter (e.g., a U6 promoter). In some embodiments, the vector can further include a coding sequence of a reporter gene (e.g., a fluorescent protein (e.g., green fluorescent protein (GFP), red fluorescent protein (RFP)) and a promoter to drive the expression of the reporter gene (e.g., a human cytomegalovirus (CMV) promoter). In some embodiments, the promoter is a native promoter, a constitutive promoter, an inducible promoter (e.g., tetracycline inducible system with a tetracycline-controlled transactivator (tTA)/reverse tTA (rtTA); Cre recombinase/loxP system), and/or a tissue specific promoter (e.g., a brain tissue specific promoter (e.g., prepro-gastrin-releasing peptide (Lebacq-Verheyden et al. (1990: Molecular Brain Research 7(3): 235-241; Costessi et al. (2006) Nucleic Acids Res 34(1): 243-253; and Boulaire et al. (2009) Adv Drug Deliv Rev 61(7-8): 589-602)). In some embodiments, the promoter is a RNA polymerase III promoter, including but not limited to, a H1 promoter, a U6 promoter, or a mouse U7 promoter.

A variety of different methods known in the art can be used to introduce any of the nucleic acids, vectors or compositions disclosed herein into a cell and/or tissue (e.g., a neuron, a glial cell, a striatum). Non-limiting examples of methods for introducing nucleic acid into a cell and/or tissue include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that the nucleic acids, vectors and compositions described herein can be introduced into any cell and/or tissue provided herein by, for example stereotaxic delivery.

### Methods of Treating

In one aspect, the present disclosure provides methods of treating a non-human transgenic animal having a neuroinflammatory disease (e.g., any of the neuroinflammatory disease described herein). The methods include steps of providing a therapeutic agent, providing a non-human transgenic animal (e.g., any of the non-human transgenic animals described herein), administering the therapeutic agent to the transgenic animal, and monitoring the transgenic animal for a phenomenon associated with a neuroinflammatory disease (e.g., any of the neuroinflammatory diseases described herein), wherein an improvement of the phenomenon in comparison to a control transgenic animal not exposed to the therapeutic agent indicates that the therapeutic agent is effective for treating the neuroinflammatory disease.

Also provided herein are methods of treating a neuroinflammatory disease that include: administering a therapeutic agent to a non-human transgenic animal overexpressing a short hairpin RNA in the brain, wherein the non-human transgenic animal has a neuroinflammatory disease, to thereby treat the neuroinflammatory disease.

In some embodiments, the neuorinflammatory disease is selected from the group consisting of: traumatic brain injury, Alzheimer's Disease, Parkinson's Disease, progressive multifocal leukoencephalopathy, and multiple sclerosis.

In some embodiments of any of the methods of treatment described herein, the method can result in increasing the life span of a subject (e.g., as compared to a similar subject having a similar neuroinflammatory disease, but receiving a different treatment).

In some embodiments of any of the methods of treatment described herein, the method results in an improvement in the motor function of the subject (e.g., as compared to the motor function of the subject prior to treatment).

In some embodiments of any of the methods of treatment described herein, the method results in delaying disease progression, reducing neuroinflammation, reducing white matter degeneration, reducing neurodegeneration, or any combination thereof.

In some embodiments of any of the methods of treatment described herein, the method can further include administering to the subject a therapeutic agent that alleviates a negative side effect of another therapeutic agent (e.g., weight loss, weight gain, lethargy).

In some embodiments, successful treatment of a neuroinflammatory disease (e.g., multiple sclerosis, Alzheimer's disease, Parkinson's disease, progressive multifocal leukoencephalopathy) can be determined in a subject using any of the conventional functional tests (e.g., mobility tests) known in the art. Non-limiting examples of mobility tests include: cylinder test, home cage observation, adhesive removal test, or inverted grid test. Mobility is compared to a control subject (e.g., a healthy age-matched subject that has not been identified as having a neuroinflammatory disease, a non-human transgenic animal that has not received at least one dose of a therapeutic agent). Improvements in mobility include an enhanced amount of movement, an enhanced speed, and/or a delayed onset of impaired mobility.

In some aspects, successful treatment of a neuroinflammatory disease can be determined by comparing the level of NF-L, Iba-1, and/or vascular permeability to a reference level of a sample obtained from a subject that has not been identified as having a neuroinflammatory disease.

In some aspects, successful treatment of a neuroinflammatory disease can be determined by performing computed tomography (CT scan), positron emission tomography (PET scan), single-photon emission computed tomography, diffuse optical imaging, magnetic resonance imaging (MM), functional magnetic resonance imaging (fMRI), electroencephalography (EEG), magnetoencephalography (MEG), near infrared spectroscopy (NIRS), optical coherence tomography, cranial ultrasound, or any combination of imaging techniques, and comparing the image to a reference image or control image obtained from a subject that has not been identified as having a neuroinflammaotry disease, or from a time point before the subject received at least one dose of a therapeutic agent.

In some embodiments of any of the methods described herein, treating can result in a decrease in Iba-1 expression and/or activity of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or between 1% and 100%, between 1% and 50%, between 1% and 25%, between 1% and 10%, between 1% and 5%, between 10% and 100%, between 10% and 50%, between 10% and 40%, between 10% and 30%, between 10% and 25%, between 10% and 20%, between 15% and 100%, between 15% and 75%, between 15% and 50%, between 15% and 40%, between 15% and 30%, between 15% and 20%, between 20% and 100%, between 20% and 75%, between 20% and 50%, between 20% and 40%, between 20% and 30%, between 25% and 100%, between 25% and 75%, between 25% and 50%, between 25% and 30%, between 40% and 100%, between 40% and 75%, between 40% and 50%, between 50% and 100%, between 50% and 75%, between 75% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, or 100%) e.g., as compared to a reference level (e.g., any of the exemplary reference levels described herein).

Non-limiting methods of determining Iba-1 include immunohistochemistry, immunofluorescence, and in situ RNA hybridization.

In some embodiments of any of the methods described herein, treating can result in an increase in NF-L expression and/or activity and/or an increase in MBP expression and/or activity of at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, at least 110%, at least 115%, at least 120%, at least 140%, at least 150%, at least 175%, at least 200%, at least 250%, at least 300%, at least 350%, at least 400%, at least 450%, at least 500%, or between 1% and 500%, between 1% and 450%, between 1% and 400%, between 1% and 350%, between 1% and 300%, between 1% and 250%, between 1% and 200%, between 1% and 150%, between 1% and 100%, between 1% and 50%, between 1% and 25%, between 1% and 10%, between 10% and 500%, between 10% and 400%, between 10% and 300%, between 10% and 200%, between 10% and 100%, between 10% and 50%, between 50% and 500%, between 50% and 400%, between 50% and 300%, between 50% and 200%, between 50% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, 100%, 105%, 110%, 115%, 120%, 125%, 130%, 140%, 150%, 160%, 170%, 180%, 190%, 200%, 250%, 300%, 350%, 400%, 450%, or 500%), e.g., as compared to a reference level (e.g., any of the exemplary reference levels described herein).

In some embodiments of any of the methods described herein, treating can reduce the vascular and/or microvascular permeability in the brain (e.g., reduce permeability of the blood brain barrier). Vascular permeability can be measured by determining the extent of vascular leakage, extravasation of antibody into the brain of a subject. Methods of determining vascular permeability are known in the art and are described herein. For example, vascular permeability can be measured by Evans blue dye extrusion, endothelial barrier antigen (EBA) staining, and monoclonal antibody leakage (e.g., radiolabeled monoclonal antibody leakage).

In some embodiments of any of the methods described herein, treating can reduce, prevent, or decrease additional loss of structures (e.g., disorganization of corpus callosum), cellular swelling, and apoptosis.

### Reference Levels

In some embodiments of any of the methods described herein, the reference level can be a level of Iba-1 in a similar sample obtained from a subject (e.g., a subject that has not been identified as a neuroinflammatory disease, or an age-matched sample obtained from a subject that is otherwise healthy). In some embodiments, a reference level can be a threshold level of Iba-1, NF-L, MBP, or a combination thereof. In some embodiments, a reference level can be a level of blood vessel permeability.

A reference level of Iba-1, NF-L and/or MBP (e.g., for tissue biopsy samples) can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the levels of Iba-1 detected in similar samples in a population of healthy subject (e.g., subjects that have not been identified as having a disease (e.g., any of the neuroinflammatory diseases described herein), do not present with a symptom of a neuroinflammatory disease, and are not considered to have an elevated risk of developing a neuroinflammatory disease).

### Therapeutic Agents

A therapeutic agent can be any type of molecule, including a small molecule, a peptide, a peptidomimetic, a polynucleotide, an antibody, a cell, or a combination thereof. Non-limiting examples of therapeutic agents that can be used in any of the methods described herein include: adenosine triphosphate, donepezil, an acetylcholinesterase inhibitor (e.g., Aricept, Exelon, Razadyne), riluzole, piromelatine, JNJ-54861911, ASP0777, radiation therapy, natalizumab (Tysabri), mefloquine, topotecan, adoptive cellular immunotherapy, physical therapy, stem cell therapy, a muscle relaxant (e.g., baclofen, tizanidine), a corticosteroid (e.g., methylprednisolone, prednisone), ubilituximab, ponesimod, teriflunomide (Aubagio), ocrelizumab, glatiramer acetate (Copaxone), dimethyl fumarate (Tecfidera), fingolimod (Gilenya), alemtuzumab (Lemtrada), mitoxantrone, an interferon beta agonist (e.g., avonex, betaseron, extavia, plegridy, rebif), a dopamine agonist (e.g., pramipexole (Mirapex), ropinirole (Requip), rotigotine (Neupro), apomorphine (Apokyn), a monoamine oxidase-B inhibitor, and a catechol-O-methyltransferase (COMT) inhibitor.

In some aspects, the therapeutic agent is a disease-modifying agent. In other aspects, the therapeutic agent alleviates and/or ameliorates symptoms associated with a neurological disease (e.g., muscle stiffness, tremors, spasms, fatigue, impaired balance, anxiety, bradykinesia, depression).

A therapeutic agent can be administered to a subject by various routes of injection including, orally, intravenously, intramuscularly, subcutaneously, intraperitoneally, intrarectally, intranasally, sublingually, or any combination of known administration methods.

Administering may be performed, e.g., at least once (e.g., at least 2-times, at least 3-times, at least 4-times, at least 5-times, at least 6-times, at least 7-times, at least 8-times, at least 9-times, at least 10-times, at least 11-times, at least 12-times, at least 13-times, or at least 14-times) a week. Also contemplated are monthly treatments, e.g., administering at least one per month for at least 1 month (e.g., at least two, three, four, five, or six or more months, e.g., 12 or more months).

### Methods of Determining the Efficacy of a Therapeutic Agent

Provided herein are methods of determining the efficacy of a therapeutic agent in a non-human transgenic animal that include: determining a first level of neurofilament light chain (NF-L), myelin basic proteins (MBP), ionized calcium-binding adaptor molecule 1 (Iba-1), or a combination thereof, in a first sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of NF-L, MBP, Iba-1, or a combination thereof, in a second sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having (1) an increased second level of NF-L, MBP, or both, as compared to the first level of NF-L, MBP, or both; and/or (2) a decreased second level of Iba-1 as compared to the first level of Iba-1. Provided herein are methods of determining the efficacy of a therapeutic agent in a non-human transgenic animal that include: determining a first level of neurofilament light chain (NF-L), myelin basic proteins (MBP), or both, in a first sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of NF-L, MBP, or both, in a second sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having an increased second level of NF-L, MBP, or both, as compared to the first level of NF-L, MBP, or both.

In some embodiments of any of the methods described herein, the method further includes determining a first level of ionized calcium-binding adaptor molecule 1 (Iba-1) in the first sample, determining a second level of Iba-1 in the second sample, and identifying the therapeutic agent as being effective in a non-human transgenic animal having a decreased second level of Iba-1 as compared to the first level of Iba-1. Also provided herein are methods of determining the efficacy of a therapeutic agent in a non-human transgenic animal that include: determining a first level of ionized calcium-binding adaptor molecule 1 (Iba-1) in a sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of Iba-1 in a sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having a decreased second level of Iba-1 as compared to the first level of Iba-1. In some embodiments of any of the methods described herein, the method further includes determining a first level of NF-L, MBP, or both, in the first sample, determining a second level of NF-L, MBP, or both, in the second sample, and identifying the therapeutic agent as being effective in a non-human transgenic animal having an increased second level of NF-L, MBP, or both, as compared to the first level of NF-L, MBP, or both.

In some embodiments, of any of the methods described herein, the method further includes determining the vascular permeability of a tissue sample at a second time point, and identifying the therapeutic agent as being effective in a non-human transgenic animal having no loss in vascular permeability.

In some embodiments, a therapeutic agent is determined to be effective when the second level of Iba-1 is decreased by at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or between 1% and 100%, between 1% and 50%, between 1% and 25%, between 1% and 10%, between 1% and 5%, between 10% and 100%, between 10% and 50%, between 10% and 40%, between 10% and 30%, between 10% and 25%, between 10% and 20%, between 15% and 100%, between 15% and 75%, between 15% and 50%, between 15% and 40%, between 15% and 30%, between 15% and 20%, between 20% and 100%, between 20% and 75%, between 20% and 50%, between 20% and 40%, between 20% and 30%, between 25% and 100%, between 25% and 75%, between 25% and 50%, between 25% and 30%, between 40% and 100%, between 40% and 75%, between 40% and 50%, between 50% and 100%, between 50% and 75%, between 75% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, or 100%) as compared to the first level of Iba-1.

In some embodiments, a therapeutic agent is determined to be effective when the second level of NF-L is increased by at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, at least 110%, at least 115%, at least 120%, at least 140%, at least 150%, at least 175%, at least 200%, at least 250%, at least 300%, at least 350%, at least 400%, at least 450%, at least 500%, or between 1% and 500%, between 1% and 450%, between 1% and 400%, between 1% and 350%, between 1% and 300%, between 1% and 250%, between 1% and 200%, between 1% and 150%, between 1% and 100%, between 1% and 50%, between 1% and 25%, between 1% and 10%, between 10% and 500%, between 10% and 400%, between 10% and 300%, between 10% and 200%, between 10% and 100%, between 10% and 50%, between 50% and 500%, between 50% and 400%, between 50% and 300%, between 50% and 200%, between 50% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, 100%, 105%, 110%, 115%, 120%, 125%, 130%, 140%, 150%, 160%, 170%, 180%, 190%, 200%, 250%, 300%, 350%, 400%, 450%, or 500%) as compared to the first level of NF-L.

In some embodiments, a therapeutic agent is determined to be effective when the second level of MBP increased by at least 1% (e.g., at least 2%, at least 4%, at least 6%, at least 8%, at least 10%, at least 12%, at least 14%, at least 15%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, at least 110%, at least 115%, at least 120%, at least 140%, at least 150%, at least 175%, at least 200%, at least 250%, at least 300%, at least 350%, at least 400%, at least 450%, at least 500%, or between 1% and 500%, between 1% and 450%, between 1% and 400%, between 1% and 350%, between 1% and 300%, between 1% and 250%, between 1% and 200%, between 1% and 150%, between 1% and 100%, between 1% and 50%, between 1% and 25%, between 1% and 10%, between 10% and 500%, between 10% and 400%, between 10% and 300%, between 10% and 200%, between 10% and 100%, between 10% and 50%, between 50% and 500%, between 50% and 400%, between 50% and 300%, between 50% and 200%, between 50% and 100%, or 1%, 2%, 3%, 4%, 5%, 6%, 8%, 10%, 12%, 14%, 15%, 16%, 18%, 20%, 22%, 24%, 25%, 26%, 28%, 30%, 35%, 40%, 45%, 50%, 55%, 60%, 65%, 70%, 75%, 80%, 85%, 90%, 95%, 100%, 105%, 110%, 115%, 120%, 125%, 130%, 140%, 150%, 160%, 170%, 180%, 190%, 200%, 250%, 300%, 350%, 400%, 450%, or 500%) as compared to the first level of MBP.

Also provided herein are methods of determining the efficacy of a therapeutic agent in delaying neuroinflammatory disease progression that include: determining a first level of ionized calcium-binding adaptor molecule 1 (Iba-1) in a sample comprising cerebrospinal fluid, blood or tissue obtained from a non-human transgenic animal at a first time point, wherein the non-human transgenic animal overexpresses a short hairpin RNA in the brain and has a neuroinflammatory disease; determining a second level of Iba-1 in a sample comprising cerebrospinal fluid, blood or tissue obtained from the non-human transgenic animal at a second time point, wherein the non-human transgenic animal received at least one dose of a therapeutic agent between the first time point and the second time point; and identifying the therapeutic agent as being effective in a non-human transgenic animal having a second level of Iba-1 that is not substantially increased as compared to the first level of Iba-1.

In some embodiments, the method further includes determining a first level of NF-L, MBP, or both, in the first sample, determining a second level of NF-L, MBP, or both, in the second sample, and identifying the therapeutic agent as being effective in a non-human transgenic animal having a second level of NF-L, MBP, or both, that is not substantially decreased as compared to the first level of NF-L, MBP, or both.

In some embodiments of any of the methods described herein, the method further includes administering one or more additional doses (e.g., at least two, at least three, at least four, at least five, at least six, at least seven, at least eight, at least nine, at least ten, or at least twelve doses) of the therapeutic agent identified as being effective to the non-human transgenic animal.

In some embodiments of any of the methods described herein, the non-human transgenic animals has previously been administered a different therapeutic agent, and the different therapeutic agent was determined not to be therapeutically effective.

In some embodiments of any of the methods described herein, the neuorinflammatory disease is selected from the group consisting of: traumatic brain injury, Alzheimer's disease, Parkinson's disease, progressive multifocal leukoencephalopathy, and multiple sclerosis.

In some embodiments of any of the methods described herein, the determining step includes using a method selected from the group consisting of: immunohistochemistry, immunofluorescence, an array-based method, Western blotting, and combinations thereof.

### EXAMPLES

The invention is further described in the following examples, which do not limit the scope of the invention described in the claims.

### Example 1—Materials and Methods

### D1shRNA Target Sequences

The target sequence of the first Drd1 shRNA was chosen according to in vitro studies [19]. The other two D1shRNAs were designed by in silico selection. Two web based siRNA selection programs were used for the design of shRNAs (MIT: http://jura.wi.mit.edu/bioc/siRNAext/; and U-Tokyo: http://alps3.gi.k.u-tokyo.acjp/˜yamada/sidirect2/index.php?type=fc). Analysis of potential “off-target” effects for each D1shRNA was conducted at the MIT web site (http://jura.wi.mit.edu/bioc/siRNAext/). The second D1shRNA2 target region was chosen from 1024 to 1046 of Drd1 mRNA, 5′ GGCCCTTGGATGGCAATTTTACT 3′ (SEQ ID NO: 9). The third D1shRNA3 target region was chosen from 2778 to 2800 of Drd1 mRNA, 5′ AAGAGCATATGCCACTTTGTATT 3′ (SEQ ID NO: 10). These shRNAs were selected due to their relatively low potential off-target effects in mouse genome, particularly at position 2 to 13 of the guide strand. The potential off-target effects for each shRNA have also been analyzed in silico, and no common unintended genes were found.

Construction of Recombinant scAAV8 Virus.

To minimize potential toxicity of fluorescence marker protein, EGFP was replaced with humanized renilla green fluorescent protein (hrGFP). The CMV-hrGFP cassette was released from pAAV-CMV-hrGFP (kindly provided by Dr. Beverly Davidson) by double digestion with EcoR I and Xba I enzymes, and was used to replace CMV-EGFP cassette in scAAV vector pVm-CMV-EGFP digested with EcoR I and Xho I enzymes. The resultant scAAV-hrGFP vector was used to clone all shRNAs at the EcoR I site. Short-hairpin RNA cassette was driven by mouse U6 promoter with a loop sequence (5′ CTTCCTGTCA 3′ (SEQ ID NO: 11)). Three different D1 shRNAs (shRNA1, shRNA2, shRNA3) targeting different Drd1 regions were cloned under the control of mouse U6 promoter. AAV8 help plasmid (kindly provided by University of Pennsylvania) was used for the generation of recombinant scAAV8 virus. The titers of the four scAAV8 virus were about 2×10¹².

### Mouse Strain and Surgery

Male C57BL/6J adult mice were purchased from Jackson Laboratory. Mice were housed in a climate-controlled animal colony with a reversed day/night cycle. Food (Harlan Teklab, Madison, Wis.) and water were available ad libitum. After a week of acclimation, 10 to 20 mice were grouped for injection of each scAAV8 viruses (control, shRNA1, shRNA2 and shRNA3). The virus was injected at both dorsal (Bregma+0.86, M/L+−1.65, D/V−2.45 mm) and ventral striatum (Bregma+0.86, M/L+−1.65, D/V −3.8) in 3-month old C57BL/6J male mice. One microliter volume of scAAV8 virus was injected slowly (2 minutes) using stereotaxic delivery at each site. The surgery and other procedures were approved by both the UCSD Animal Care and Use Committee (UCSD Animal Use Protocol: S04190M) and local Veteran's Administration Hospital (Animal Use Protocol: 04-036 (VAH)) prior to the onset of the experiments. Mice were maintained in American Association for Accreditation of Laboratory Animal Care approved animal facilities. This facility meets all Federal and State requirements for animal care.

### Western Blot and Immunohistochemistry

Mice were anesthetized with carbon dioxide, and the brains were quickly removed on ice. Both anterior and posterior striatum were dissected form coronal brain sections on ice. Tissues were homogenized using Dounce Homogenizer in 1× Passive Lysis Buffer (Promega) supplemented with Protease Inhibitor Cocktail (Sigma). Protein concentration was measured with Bradford Assay Kit (Pierce). Twenty microgram of total proteins were loaded on SDS-PAGE gel, and blotted to PVDF membrane (Bio-Rad) after separation with electrophoresis. Mouse monoclonal antibody anti-Drd1 (abcam) and mouse monoclonal antibody anti-NMDAR1 (BD Biosciences) were used as primary antibodies. HRP conjugated anti-mouse IgG and HRP conjugated anti-rabbit IgG, were obtained from Amersham and Sigma as the secondary antibodies. The concentration of primary antibodies for Western Blot was generally used as recommended by the manufacturers. Immunohistochemical staining was performed as previously described [20]. Mouse monoclonal antibody anti-Drd1 (abeam), rabbit anti-Drd1 (Sigma), rabbit anti-Iba-1 (Wako, 019-19741); rabbit anti-MBP (Dako, #A0623), and goat anti-NF-L (C-15, Santa Cruz, #sc-12980) were used as primary antibodies. Mouse brains were analyzed 7 weeks post-injection. In brief, paraffin brain sections were baked, deparaffinized, and rehydrated. After rinsed in distilled water, the sections were submerged in Tris-EDTA Buffer (10 mM Tris Base, 1 mM EDTA Solution, 0.05% Tween 20, pH 9.0), and autoclaved (Tuttnauer, 2340 M) for antigen retrieval at 121° C. for 20 minutes. After autoclaving, endogenous peroxidase activity was quenched with incubation of 0.3% hydrogen peroxide in PBS for 30 minutes at room temperature. The slides were washed and blocked with 2.5% normal horse serum (ImmPRESS, Vector Labs, S-2012). After incubation with the primary antibodies, ImmPRESS peroxidase-micropolymer conjugated horse anti-mouse, anti-rabbit, anti-goat IgG (Vector Labs) antibodies were used as the secondary antibody. Chromogenic reaction was conducted with ImmPACT NovaRED Peroxidase Substrate (Vector Labs, SK-4805) for 5 minutes with rotational shaking. After staining, the slides were washed and air-dried overnight. Next day, the slides were mounted with Cytoseal 60 mounting medium (Richard-Allan Scientific, 8310-16).

### Example 2—Massive Mouse IgG Leakage in Brain Tissue Transduced with scAAV8-D1shRNAs

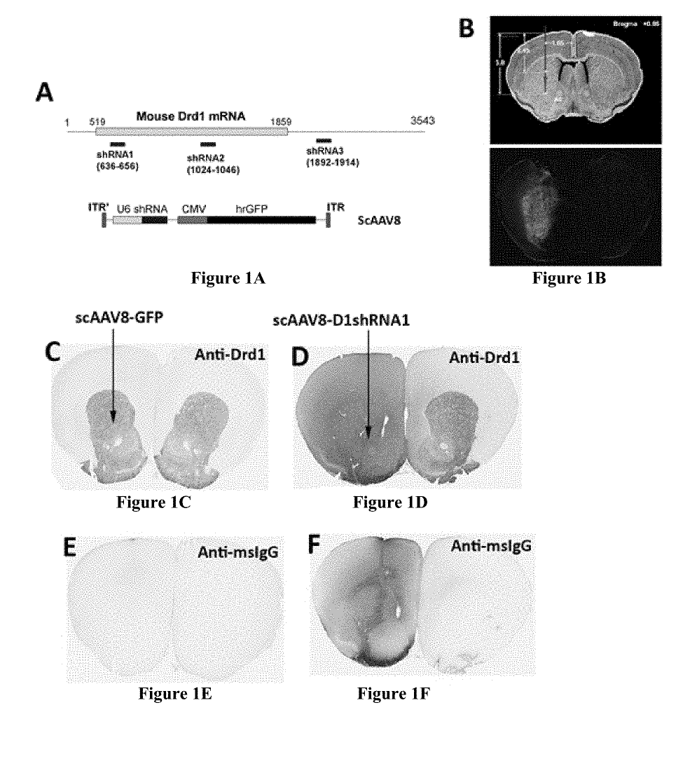

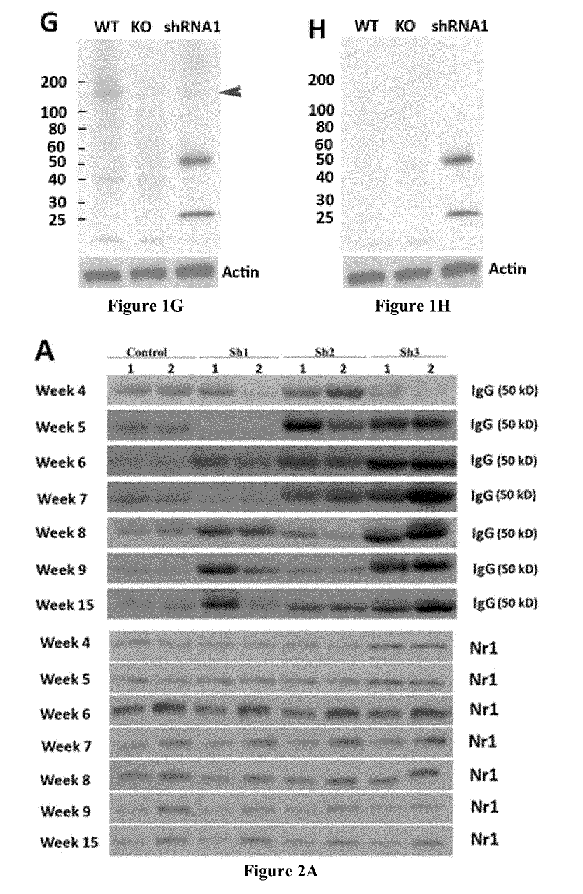

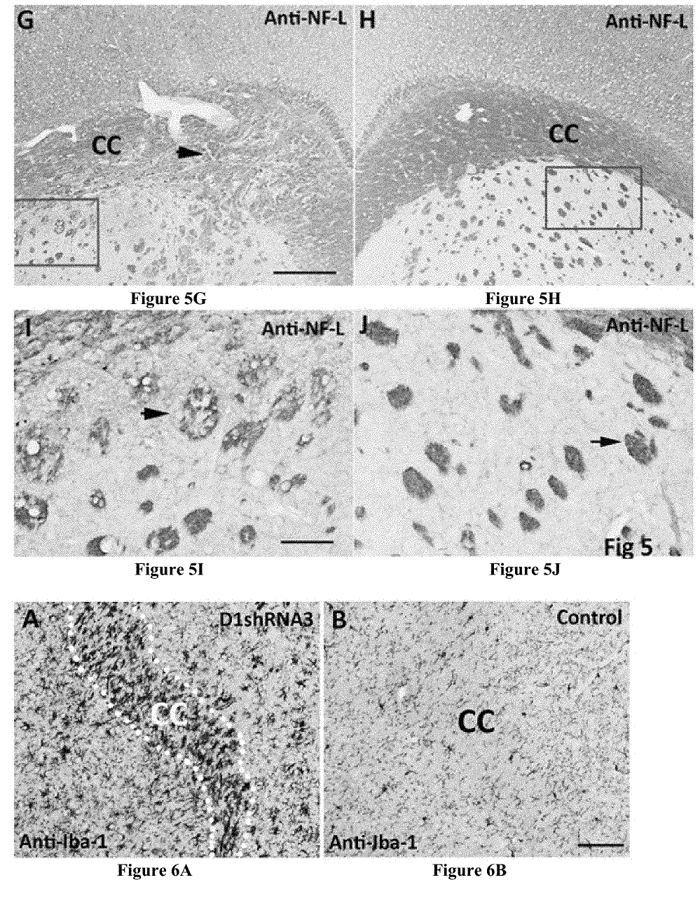

We initially planned to investigate striatal Drd1 functions in modulation of behaviors related to psychiatric disorders. To silence Drd1 expression, 3 different short hairpin RNAs (shRNA) were designed that have lengths ranging from 21 to 23 bp to limit double-stranded RNA (dsRNA) induced innate immune responses [1]. Both the D1shRNA1 and the D1shRNA2 were designed to target the coding sequence of mouse Drd1 gene (SEQ ID NO: 1 and 3), and the D1shRNA3 (SEQ ID NO: 5) was selected to target the 3′ UTR of Drd1 gene (FIG. 1A, 7A). All three D1shRNAs were driven by mouse U6 promoter in scAAV8 (self-complementary AAV) shuttle vector expressing humanized renilla green fluorescent protein (hrGFP). Recombinant scAAV8-hrGFP virus was generated as the control. Using stereotaxic delivery, each scAAV8 virus was injected into mouse striatum (FIG. 1B). Four weeks after injection, hrGFP expression was examined for transduction efficiency. Consecutive cryostat sections (from Bregma 1.86 to −0.86) confirmed that scAAV8 transduced a high volume of mouse striatum [21]. To evaluate suppression of Drd1 protein in striatum, immunohistochemical staining of brain paraffin sections from both the control hrGFP and the D1shRNA1 mice was conducted. Drd1 protein was readily detected in mouse striatum by using mouse anti-Drd1 antibody (FIG. 1C). Surprisingly, the antibody generated extensive background across the left hemisphere transduced with scAAV8-D1shRNA1, but not in the right hemisphere without virus injection (FIG. 1D). Adjacent brain sections were then immunostained with anti-mouse IgG secondary antibody only. The secondary antibody did not generate signals in mouse brain transduced with scAAV8-hrGFP (FIG. 1E). However, the secondary antibody detected extensive mouse IgG signals in the left hemisphere transduced with scAAV8-D1shRNA1, but not in the uninjected right hemisphere (FIG. 1F), suggesting that there is extensive mouse IgG leakage from blood capillaries into the left brain hemisphere. Western blot was performed to detect both Drd1 protein suppression and the presence of mouse IgG in mouse striatum (FIG. 1G). Drd1 protein was reduced (arrowhead) in the striatum transduced with D1shRNA1, and is absent in the striatum of Drd1 knockout mice. Reduction of striatal Drd1 protein was also confirmed in immunnohistochemical analysis using rabbit anti-Drd1 antibody in the striatum transduced with scAAV8-D1shRNA1 (FIG. 7B-E). Anti-rabbit IgG secondary antibody cannot recognize leaked mouse IgG in the left brain hemisphere. Consistent with immunohistochemical analysis of mouse IgG leakage to brain tissue, mouse IgG heavy (50 kD) and light chains (25 kD) was observed in Western blot analysis using mouse anti-Drd1 antibody (FIG. 1G). Western blot analysis using only anti-mouse IgG secondary antibody confirmed that these two bands are indeed mouse IgG heavy and light chains (FIG. 1H). To examine whether high levels of leaked mouse IgG are also present in mouse striatum transduced by other D1shRNAs, Western blot analyses of mouse striatum transduced with either the control hrGFP or each D1shRNA virus at different post-injection time points was performed (FIG. 2A). Mouse IgG levels were remarkably increased in the striatum of mice injected with all scAAV8-D1shRNAs, particularly scAAV8-D1shRNA3, in comparison with the control mice injected with scAAV8-hrGFP. Longitudinal studies from week 4 to week 15 revealed persistent excessive mouse IgG antibodies in striatum for several months. After normalization against the control in each Western blot, all mouse IgG data was combined (see FIG. 2B). Striatum transduced with scAAV8-D1shRNA3 virus had a significantly higher level of IgG (Student's t-test, p<0.01) than the control striatum transduced with scAAV8-hrGFP. Many mice injected with either scAAV8-shRNA1 or scAAV8-shRNA2 virus also displayed excessive mouse IgG in their striatum. Consistent with the Western blot, almost all mice injected with scAAV8-D1shRNAs displayed IgG background in immunohistochemical staining with anti-mouse IgG antibodies (Table 1).

TABLE 1
IgG Immunostaining in Individual Mouse Striatum

| | Mouse IgG Intensities (IHC) | | | | |
|---|---|---|---|---|---|
| | | | | No. of | Percentage |
| | Negative | +++ | ++ | mice | of mice (%) |
| scAAV8-GFP | 6 | 0 | 0 | | 0 |
| scAAV8- | 0 | 6 | 3 | 6 | 100 |
| D1shRNA1 | | | | | |
| scAAV8- | 1 | 2 | 4 | 10 | 90 |
| D1shRNA2 | | | | | |
| scAAV8- | 1 | 3 | 2 | 10 | 88 |
| D1shRNA3 | | | | | |

No differences in IgG staining were observed between the left injected striatum and the right control striatum in scAAV8-hrGFP mice. A low level of striatal IgG detected in scAAV8-hrGFP mice by the Western blot, but not by the immunohistochemical staining, may be caused by different sensitivities of the two techniques and individual mouse variability.

### Example 3—Neuroinflammation and Microglial Activation

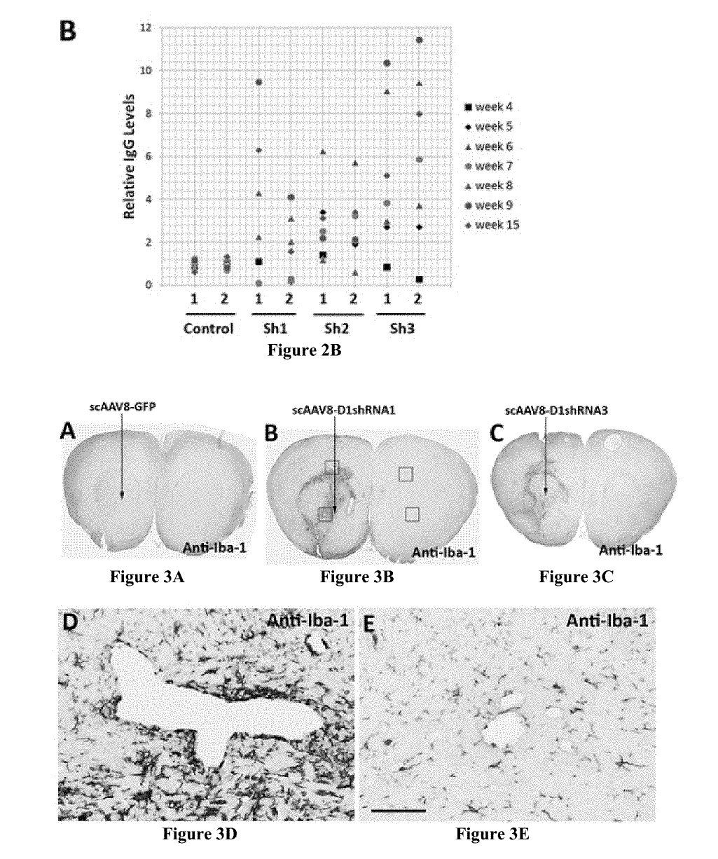

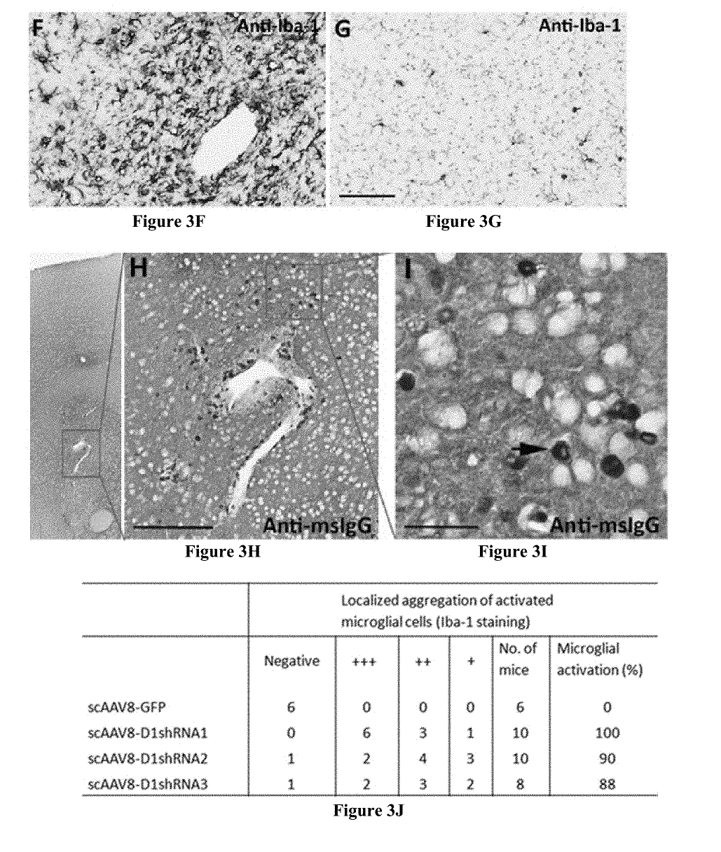

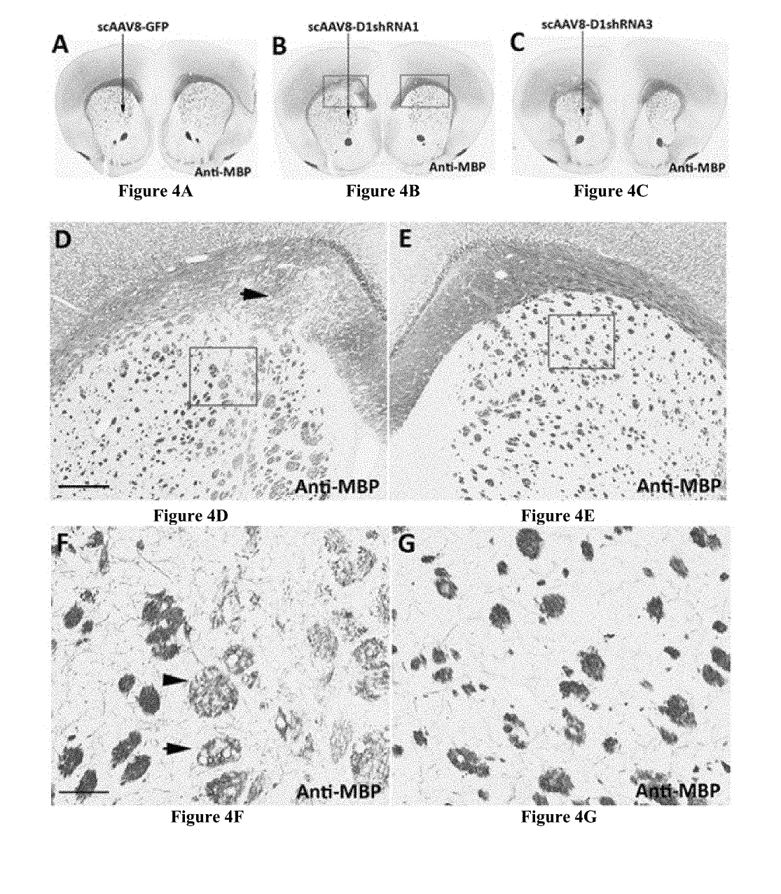

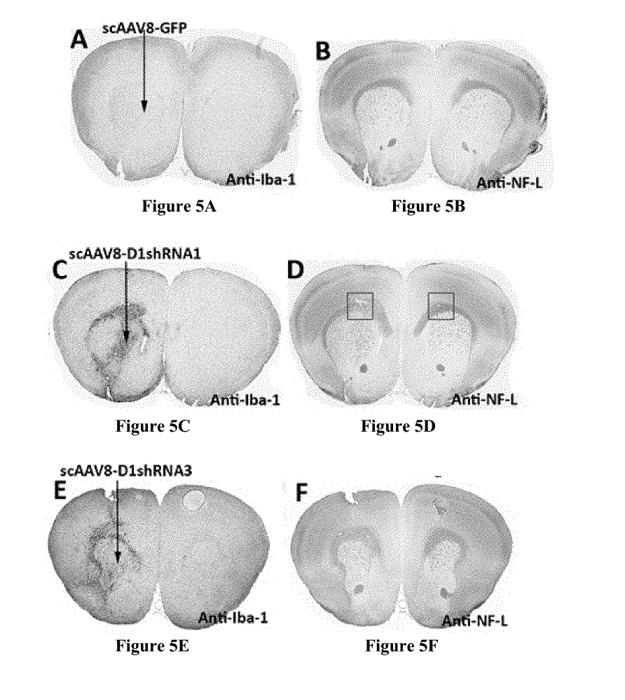

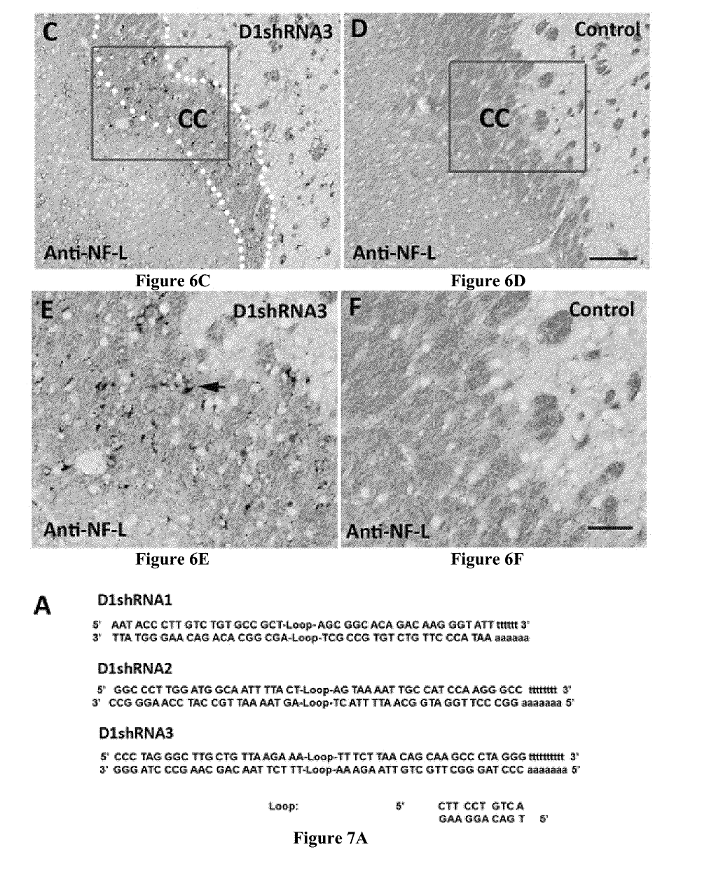

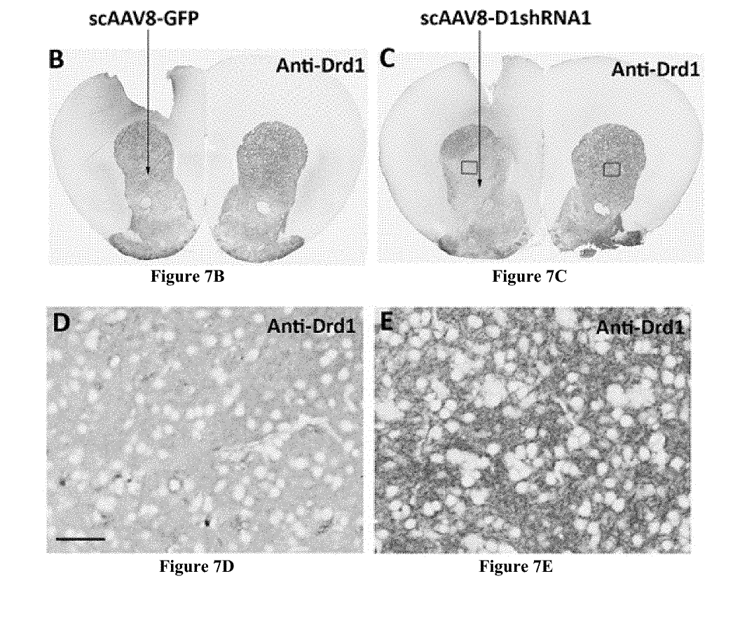

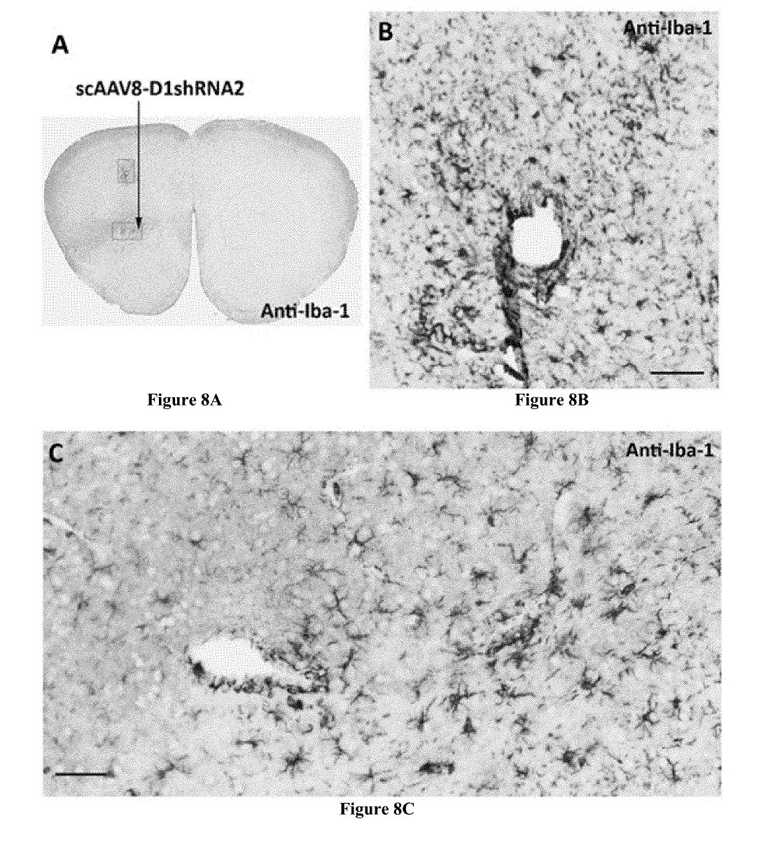

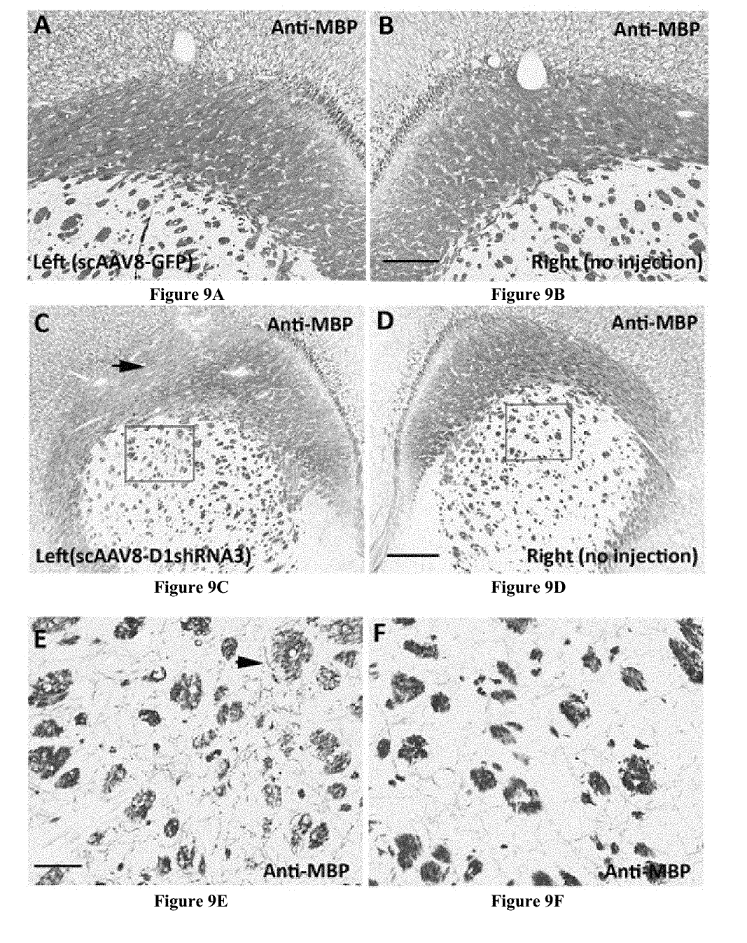

To determine whether excessive mouse IgG leakage in striatum indicated neuroinflammation, microglial activation using immunohistochemical staining of Iba-1 protein was tested. Microglial cells were extensively over-proliferated and hypertrophied in the striatum transduced with scAAV8-D1shRNAs, but not scAAV8-hrGFP virus (FIG. 3A-3C). These data suggest that D1shRNA, but not the scAAV8 virus, causes neuroinflammation. Consistent with neuroinflammation, blood capillaries were surrounded by activated microglial cells and markedly swollen (FIG. 3D-3G). Peripheral immune cells intensively labeled with IgG infiltrated into the inflammatory brain tissue surrounding a swollen capillary, indicating a breakdown of blood brain barrier (FIGS. 3H and 3I). Such an intensive neuroinflammation is likely responsible for excessive mouse IgG leakage to the striatum transduced with scAAV8-D1shRNAs. These IgG antibodies may come from either leakage of blood brain barriers or be produced by infiltrated B cells in the inflammatory brain tissues [27, 33, 34]. Persistent excessive IgG antibodies indicate chronic inflammation. The extent of neuroinflammation in individual mice transduced with different scAAV8-D1shRNAs was examined using anti-Iba-1 immunostaining as a marker (FIG. 3H). Massive activation of microglial cells in FIGS. 3B and 3C was arbitrarily ranked as “+++”, and localized mild microglial activation in FIG. 8 was ranked as “+”. Mice were examined blind to scAAB8 virus genotype. After analyzing 34 individual mice injected with either scAAV8-hrGFP or scAAV8-D1shRNAs, microglial activation was not detected in any control mouse brain (n=6) transduced with scAAV8-hrGFP virus. Almost all mouse brains transduced with scAAV8-D1shRNA virus displayed different degrees of microglial activation (FIG. 3H). The activated microglial cells were particularly concentrated on corpus callosum (FIGS. 3B and 3C), the largest cerebral white matter. To investigate whether there is white matter degeneration in corpus callosum, immunohistochemical staining of myelin basic proteins (MBP) was conducted. There was no difference in MBP staining between the left striatum transduced with scAAV8-hrGFP virus (FIG. 9A) and the right striatum without virus injection (FIG. 9B). In comparison with the control mice (FIG. 4A), decreased MBP staining was observed in the corpus callosum on the left side transduced with either scAAV8-D1shRNA1 (FIG. 4B) or scAAV8-D1shRNA3 (FIG. 4C). Under a higher magnification, localized swelling and disorganization of corpus callosum was observed in the left striatum transduced with scAAV8-D1shRNA1 (FIG. 4D) in comparison with the right striatum without virus injection (FIG. 4E). In addition to corpus callosum, striatal white matter tracts were also degenerated in the left striatum (FIG. 4F) in contrast to the control right striatum (FIG. 4G). To investigate whether white matter degeneration may damage inside large-caliber axons, immunohistochemical staining of neurofilament light chain (NF-L), an abundant structure protein of large-caliber axons was conducted. Two adjacent paraffin sections were immunostained with anti-Iba-1 and anti-NF-L antibodies respectively (FIG. 5A-5F). The left side corpus callosum transduced with scAAV8-shRNA1 was swollen and disorganized with reduced NF-L staining in comparison with the control right side corpus callosum (FIGS. 5G and 5H). Striatal white matter tracts were also swollen and blebbed (FIGS. 5I and 5J). None of these anatomical abnormalities were observed in mouse brain transduced with scAAV8-hrGFP virus (FIG. 10). Reduced NF-L staining was also observed in the corpus callosum transduced with scAAV8-D1shRNA3 virus (FIG. 11). Activated microglial cells (Iba-1 immunostaining) were concentrated on corpus callosum (NF-L immunostaining) on two adjacent brain sections under a higher magnification (FIG. 6A-6D). Condensed aggregates of NF-L immunostaining were observed in both corpus callosum and striatal white matter tracts (FIG. 6C-6F), suggesting that D1shRNA-induced neuroinflammation causes both white matter degeneration and injuries of inside large-caliber axons in addition to microglial activation. Next, it was determined whether NF-L in the corpus callosum of individual mice transduced with different scAAV8-D1shRNAs was reduced using anti-NF-L immunostaining as a marker (Table 2).

TABLE 2
NF-L immunostaining in corpus callosum

| | NF-L reduction in CC (IHC) | | | | | |
|---|---|---|---|---|---|---|
| | | | | | | Percentage |
| | | | | | No. of | of mice |
| | Negative | +++ | ++ | + | mice | (%) |
| scAAV8-GFP | 6 | 0 | 0 | 0 | 6 | 0 |
| scAAV8-D1shRNA1 | 1 | 4 | 4 | 1 | 10 | 90 |
| scAAV8-D1shRNA2 | 2 | 2 | 3 | 3 | 10 | 80 |
| scAAV8-D1shRNA3 | 1 | 2 | 3 | 2 | 8 | 80 |

NF-L reduction was arbitrarily ranked after within-section comparisons. None of the scAAV8-hrGFP mice displayed NF-L reduction in the injected brain hemisphere, whereas most mice injected with scAAV8-D1shRNA exhibited degrees of NF-L reduction. Despite neuroinflammation and white matter degeneration, scAAV8-D1shRNA mice were indistinguishable from the control scAAV8-hrGFP mice in either locomotion or prepulse inhibition test. No other behavioral tests were performed. Continuing deterioration of neuroinflammation and white matter degeneration may be required for the development of neurologic symptoms and behavioral abnormalities in scAAV8-D1shRNA mice. We unexpectedly found that transduction of mouse striatum with scAAV8-D1shRNA virus, but not the scAAV8 virus itself, brain injuries from stereotaxic surgeries or hrGFP over-expression, induces extensive neuroinflammation, white matter degeneration, and injuries of large-caliber axons.

Various factors, such as shRNA off-target silencing, Drd1 silencing, and shRNA immunogenicity, etc., may be involved in the development of neuroinflammation and white matter degeneration caused by scAAV8-D1-shRNAs.

shRNA Off-Target Silencing

Three D1shRNAs were designed that have different target sequences. No common potential off-target genes were found among these three D1shRNAs by in silico analyses. Therefore, it is unlikely that D1shRNA off-target silencing effects play a major role in induction of neuroinflammation.

### Drd1 Silencing

Overexpression of nonspecific AAV1/2-shRNA was reported to cause neuronal toxicity associating with microglial activation in mouse striatum [22]. However, neither white matter degeneration nor axonal injuries were reported. Importantly, not all shRNAs can induced microglial activation [22], suggesting that shRNAs have sequence-dependent but not sequence-specific effects on activating innate immune responses. Therefore, it is difficult to use nonspecific shRNAs to rule out a potential role of Drd1 silencing in activation of the innate immune system.

shRNA Immunogenicity

It is likely that D1shRNA immunogenicity plays an important role in microglial activation and white matter degeneration [22]. siRNAs/shRNAs have been well studied for eliciting innate immune responses by activating Toll-like receptor 3 (TLR-3) and/or protein kinase RNA-activated (PKR) [8, 35, 36]. TLR3 is activated by dsRNAs with a length of at least 40 to 50 bp [37]. The dsRNA stems of D1shRNA1, D2 shRNA2 and D1shRNA3 are between 21 to 23 bp that are insufficient to dimerize and activate TLR-3. PKR is localized in cytoplasm and can be activated by dsRNAs with a length of at least 30 bp [38]. However, shorter dsRNAs were reported to bind and activate PKR [8, 39, 40]. Therefore, D1shRNAs could have activated PKR in neurons and/or glial cells to initiate inflammatory responses. Sequence-dependent TLR7 was reported to mediate siRNA immunogenicity [6]. TLR7 may be another candidate downstream effector of D1shRNAs in the activation of CNS innate immune responses.

### Innate Immune Cells

Different cell tropisms between AAV1/2 and AAV8 serotypes could be an important difference between McBride et al and our studies. AAV1/2 virus preferentially transduces neuronal cells, whereas AAV8 virus transduces both neuronal cells and glial cells [23]. Transduction of glial cells, particularly microglial cells by scAAV8-D1shRNAs, could contribute to extensive neuroinflammation and white matter degeneration. Activated microglial cells were predominantly concentrated on corpus callosum and co-localized with demyelination, suggesting that the activated microglial cells were polarized to the M1 cytotoxic phenotype [41]. Oligodendrocytes are the most vulnerable CNS cells to glutamate excitotoxicity [42]. Oligodendrocytes in scAAV8-D1shRNA mice could have been damaged by excessive glutamate and thereby were attacked by activated microglial cells. Since peripheral immune cells infiltrated intro inflammatory brain tissue in scAAV8-D1shRNA mice, it was not possible to determine whether activated microglial cells were derived from infiltrated peripheral macrophages or CNS resident microglial cells. Nevertheless, CNS resident microglial cells are likely one of the early players activated by D1shRNA over-expression, which subsequently causes neuroinflammation to breakdown blood brain barrier for infiltration of peripheral immune cells including macrophages. Interactions between activated CNS innate immune cells and infiltrated peripheral adaptive immune cells exacerbate white matter degeneration. Therefore, scAAV8-D1shRNAs may have transduced striatal neurons and a variety of glial cells including microglia, astrocytes, and oligodendrocytes, which may contribute to extensive neuroinflammation and white matter degeneration. Neuroinflammation in mouse cortex was also observed where Drd1 is not expressed (FIGS. 8A and 8B). Drd1 is expressed at low levels in microglial cells and T lymphocytes, and dopamine modulates brain inflammation [24]. Therefore, suppression of Drd1 in non-neuronal cells by scAAV8-D1shRNA may contribute to the extensive neuroinflammation.

### RNA Virus and Retroviral RNA in MS

CNS demyelination can be naturally induced by a variety of virus in animals including visna virus, caprine arthritis-encephalitis virus, Theiler's murine encephalomyelitis virus, Semliki Forest virus, etc [27]. All these are RNA viruses that produce massive viral dsRNAs to activate the innate immune system in infected host cells, suggesting that human RNA viruses are candidate pathogens in some human MS patients. Recent studies demonstrated that massive dsRNA can also be produced in the absence of virus infection by transcription of repetitive genomic sequences or endogenous retroviral sequences of the genome due to aberrant epigenetic modifications, which subsequently activates the innate immune system to over-express pro-inflammatory cytokines [22, 31, 43, 44]. Chronic massive production of dsRNA/shRNA induces neuroinflammation and white matter degeneration, suggesting that epigenetic aberration can be involved in the pathogenesis of human MS and other inflammatory diseases.

### Example 4—D1shRNA Over-Expression in Hippocampus

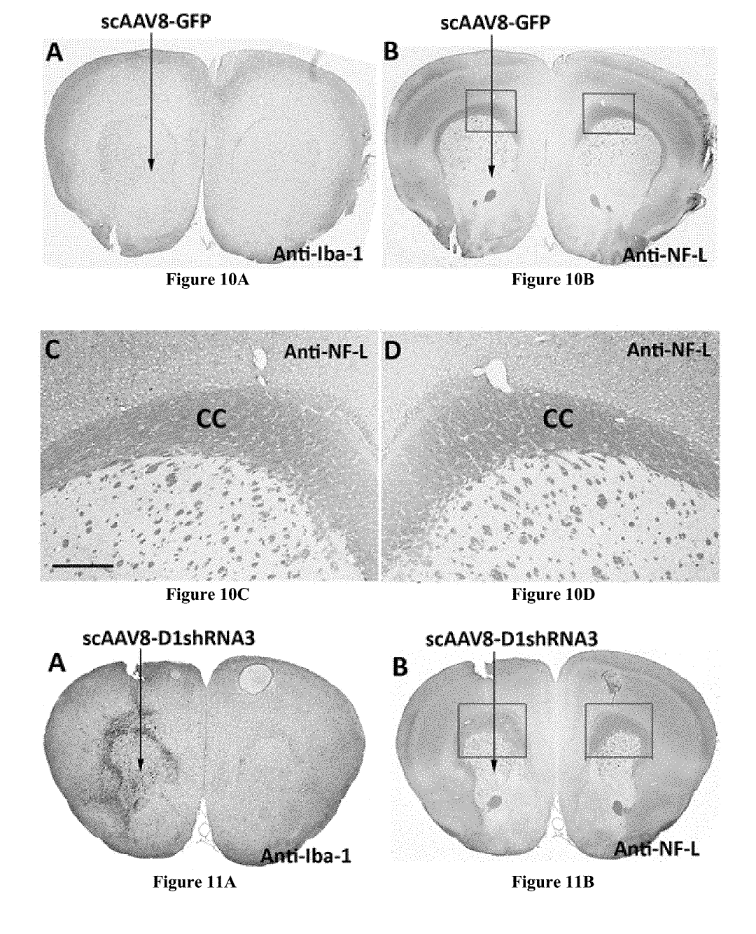

To exclude involvement of Drd1 suppression in initiation of neuroinflammation, the scAAV8-D1shRNA virus was injected into mouse hippocampal dentate gyrus (DG) where Drd1 is not expressed. Seven weeks after virus injection, neuroinflammation was examined in the hippocampus of both the control scAAV8-hrGFP and the scAAV8-D1shRNA mice. The same neuroinflammatory effects were observed in the hippocampal dentate gyrus of all 3 mice injected with scAAV8-D1shRNA viruses, but not the control scAAV8-hrGFP mice (FIGS. 12A and 12B). Neither extravasation of mouse IgG antibodies nor microglial activation was observed in mouse hippocampus injected with scAAV8-hrGFP virus (FIGS. 12A and 12B). Extravasation of IgG (IgG staining in the left DG, FIG. 12C and its inset), infiltration of IgG⁺-immune cells (perivascular space, FIG. 12C), and microglial activation (FIG. 12D) were observed in the left hippocampal dentate gyrus transduced by the scAAV8-D1shRNA virus, but not in the control right hippocampal dentate gyrus.

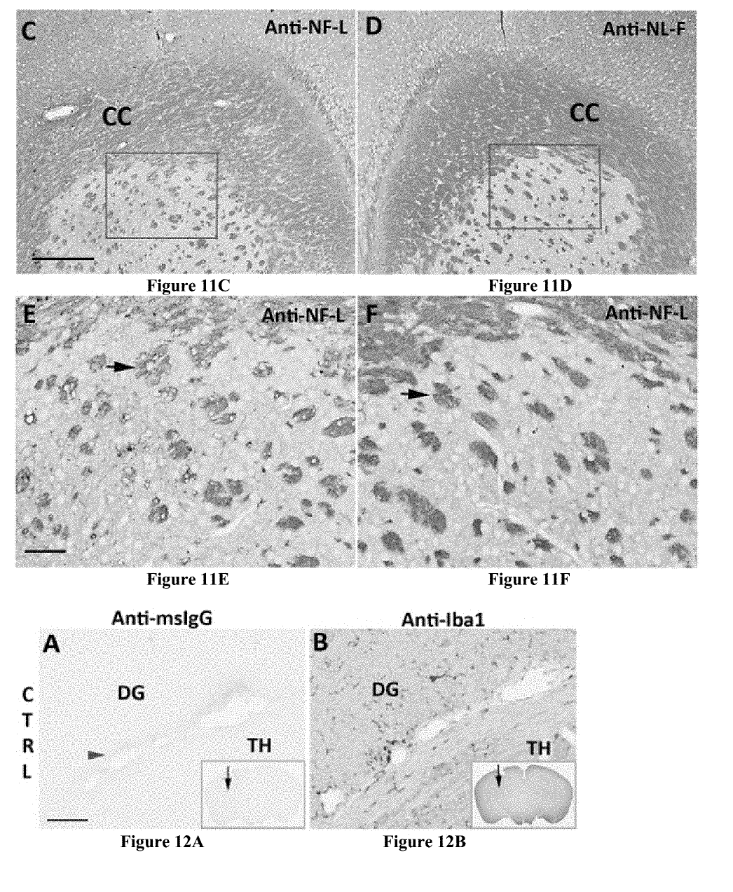

In the scAAV8-D1shRNA mice, persistent neuroinflammation caused degeneration of hippocampal dentate gyrus that is associated with both a shrinking of the thickness of its molecular layers and loss of dentate granule cells (FIG. 13A) in comparison with the right hippocampus of the same brain section (FIG. 13B). Expression of GluR1 proteins, a subunit of α-amino-3-hydroxy-5-methyl-4-isoxazolepropionic acid (AMPA) receptors, was completely abolished in the degenerating left hippocampal dentate gyrus (FIG. 13C) in comparison with the right hippocampus of the same brain section (FIG. 13D). In the control scAAV8-hrGFP mice, neither neurodegeneration nor a decrease of GluR1 proteins was observed in the left hippocampal dentate gyrus injected with the scAAV8-hrGFP virus (data not shown). Therefore, these experiments showed that D1shRNA is involved in the generation of neurinflammation in the hippocampus.

### Example 5—Over-Expression of a Random shRNA in Striatum

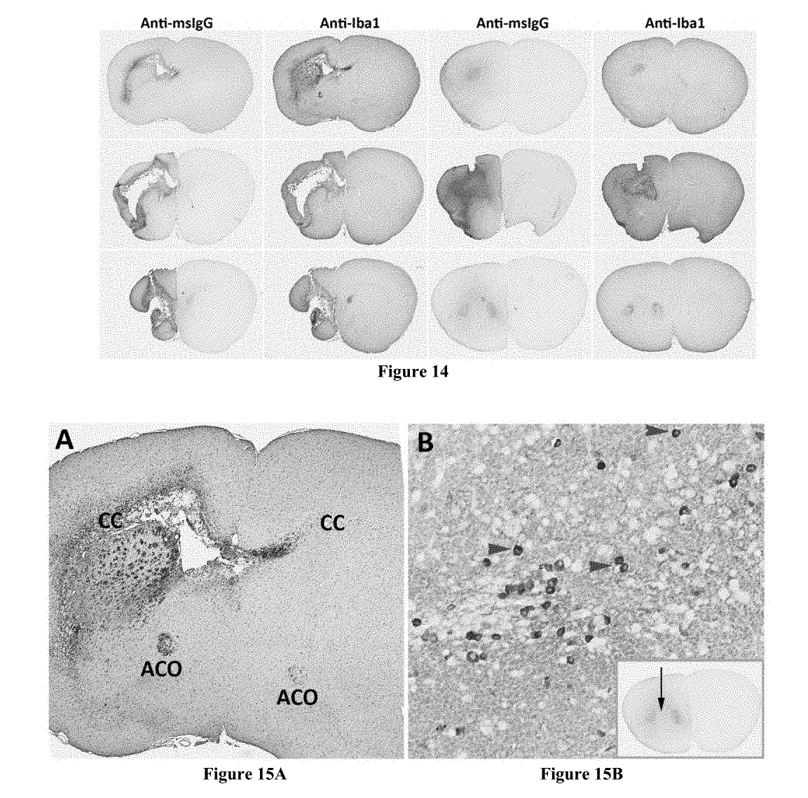

To provide definitive evidence that shRNA immunogenicity induces neuroinflammation, a random sequence shRNA was over-expressed in mouse striatum using the same scAAV8-shRNA vector. Over-expression of the random shRNA induces neuroinflammation associated with extravasation of mouse IgG antibodies and microglial activation in all 6 mice injected with scAAV8-shRNA viruses (FIG. 14). None of the control scAAV8-hrGFP mice showed any neuroinflammation (data not shown). Half of the scAAV8-shRNA mice displayed extensive white matter degeneration (particularly corpus callosum), and one of them almost lost the left hemisphere. The remaining 3 mice displayed milder neuroinflammation that is characterized with concurrent co-localized IgG extravasation and microglial activation. Under high magnification, activated microglial cells preferentially attacked white matter such as corpus callosum, striatal white matter tracts, and anterior commissure (FIG. 15A). Infiltration of peripheral immune cells was observed even in an early phase of neuroinflammation (FIG. 15B).

This experiment unequivocally demonstrated that shRNA immunogenicity, rather than its gene silencing, induces chronic, local neuroinflammation that is associated with microglial activation, extravasation of IgG, infiltration of peripheral immune cells, and brain degeneration. Neuroinflammation by shRNA immunogenicity induces the breakdown of blood brain barriers. This experiment confirms the data shown in Examples 3-4, and further indicates that any composition comprising a shRNA can induce neuroinflammation.

While striatal neuroinflammation causes predominantly white matter degeneration, hippocampal neuroinflammation causes neuronal cell degeneration. Thus, neuroinflammation generates different degenerations in different brain regions.

### Example 6—Utilities of shRNA Immunogenicity for Drug Development

shRNA could be used as a potent immunogen to induce local neuroinflammation and degeneration at any specific brain region. To further improve the technology, potency of shRNA immunogenicity could be controlled by shRNA abundance using tetracycline inducible system such that neuroinflammation intensities can be controlled by using doxycycline dosages. Therefore, either low-grade or high-grade chronic neuroinflammation can be induced and maintained at any specific brain region. Relapsing and remitting neuroinflammation can also be generated with the presence or absence of doxycycline. As shown in Examples 4 and 5, shRNAs induced neuroinflammation and degeneration in both striatum and hippocampus. Therefore, shRNA immunogenicity can be used to generate a variety of animal models to investigate neuroinflammation in the pathogenesis of neuroinflammatory diseases: multiple sclerosis, Alzheimer's Disease, Parkinson's Disease, and more. For example, a high grade neuroinflammation and white matter degeneration can be induced in either spinal cord or brain (corpus callosum) by using shRNA immunogenicity to model multiple sclerosis. As another example, a persistent low-grade neuroinflammation can be generated in hippocampus by using shRNA immunogenicity to induce neurodegeneration to model Alzheimer's Disease; and Parkinson's disease can be modeled by causing a persistent low-grade neuroinflammation can be generated in substantia nigra by using shRNA immunogenicity. Progressive multifocal leukoencephalopathy (PML), which is a rare and usually fatal viral disease caused by reactivation of JC viruses, is characterized by degeneration and inflammation of the white matter. In addition, low-grade relapsing-remitting neuroinflammation can be induced using shRNA immunogenicity such that it will not be sufficient to trigger significant neurodegeneration.

Furthermore, the animal models described herein can be to develop and test therapeutic drugs for these diseases.

### REFERENCES

1. Reynolds A, Anderson E M, Vermeulen A, Fedorov Y, Robinson K, Leake D, Karpilow J, Marshall W S, Khvorova A: Induction of the interferon response by siRNA is cell type- and duplex length-dependent. RNA 2006, 12:988-993.
2. Davidson B L, Harper S Q: Viral delivery of recombinant short hairpin RNAs. Methods Enzymol 2005, 392:145-173.
3. Harper S Q, Staber P D, He X, Eliason S L, Martins I H, Mao Q, Yang L, Kotin R M, Paulson H L, Davidson B L: RNA interference improves motor and neuropathological abnormalities in a Huntington's disease mouse model. Proc Natl Acad Sci USA 2005, 102:5820-5825.
4. Harper S Q, Davidson B L: Plasmid-based RNA interference: construction of small-hairpin RNA expression vectors. Methods Mol Biol 2005, 309:219-235.
5. Umbach J L, Cullen B R: The role of RNAi and microRNAs in animal virus replication and antiviral immunity. Genes Dev 2009, 23:1151-1164.
6. Hornung V, Guenthner-Biller M, Bourquin C, Ablasser A, Schlee M, Uematsu S, Noronha A, Manoharan M, Akira S, de Fougerolles A, et al: Sequence-specific potent induction of IFN-alpha by short interfering RNA in plasmacytoid dendritic cells through TLR7. Nat Med 2005, 11:263-270.
7. Judge A D, Sood V, Shaw J R, Fang D, McClintock K, MacLachlan I: Sequence-dependent stimulation of the mammalian innate immune response by synthetic siRNA. Nat Biotechnol 2005, 23:457-462.
8. Sledz C A, Holko M, de Veer M J, Silverman R H, Williams B R: Activation of the interferon system by short-interfering RNAs. Nat Cell Biol 2003, 5:834-839.
9. Robbins M A, Li M, Leung I, Li H, Boyer D V, Song Y, Behlke M A, Rossi J J: Stable expression of shRNAs in human CD34+ progenitor cells can avoid induction of interferon responses to siRNAs in vitro. Nat Biotechnol 2006, 24:566-571.
10. Rao D D, Vorhies J S, Senzer N, Nemunaitis J: siRNA vs. shRNA: similarities and differences. Adv Drug Deliv Rev 2009, 61:746-759.
11. McCarty D M, Fu H, Monahan P E, Toulson C E, Naik P, Samulski R J: Adeno-associated virus terminal repeat (T R) mutant generates self-complementary vectors to overcome the rate-limiting step to transduction in vivo. Gene Ther 2003, 10:2112-2118.
12. Fu H, Muenzer J, Samulski R J, Breese G, Sifford J, Zeng X, McCarty D M: Self-complementary adeno-associated virus serotype 2 vector: global distribution and broad dispersion of AAV-mediated transgene expression in mouse brain. Mol Ther 2003, 8:911-917.
13. McCarty D M, Monahan P E, Samulski R J: Self-complementary recombinant adeno-associated virus (scAAV) vectors promote efficient transduction independently of DNA synthesis. Gene Ther 2001, 8:1248-1254.
14. Gerfen C R: The neostriatal mosaic: multiple levels of compartmental organization in the basal ganglia. Annu Rev Neurosci 1992, 15:285-320.
15. Nagatsua T, Sawadab M: L-dopa therapy for Parkinson's disease: past, present, and future. Parkinsonism Relat Disord 2009, 15 Suppl 1:S3-8.
16. Gantois I, Fang K, Jiang L, Babovic D, Lawrence A J, Ferreri V, Teper Y, Jupp B, Ziebell J, Morganti-Kossmann C M, et al: Ablation of D1 dopamine receptor-expressing cells generates mice with seizures, dystonia, hyperactivity, and impaired oral behavior. Proc Natl Acad Sci USA 2007, 104:4182-4187.
17. Le Foll B, Gallo A, Le Strat Y, Lu L, Gorwood P: Genetics of dopamine receptors and drug addiction: a comprehensive review. Behav Pharmacol 2009, 20:1-17.
18. Knutson B, Gibbs S E: Linking nucleus accumbens dopamine and blood oxygenation. Psychopharmacology (Berl) 2007, 191:813-822.
19. Isacson R, Kull B, Salmi P, Wahlestedt C: Lack of efficacy of ‘naked’ small interfering RNA applied directly to rat brain. Acta Physiol Scand 2003, 179:173-177.
20. Kim M, Soontornniyomkij V, Ji B, Zhou X: System-wide immunohistochemical analysis of protein co-localization. PLoS One 2012, 7:e32043.
21. Broekman M L, corner L A, Hyman B T, Sena-Esteves M: Adeno-associated virus vectors serotyped with AAV8 capsid are more efficient than AAV-1 or -2 serotypes for widespread gene delivery to the neonatal mouse brain. Neuroscience 2006, 138:501-510.
22. McBride J L, Boudreau R L, Harper S Q, Staber P D, Monteys A M, Martins I, Gilmore B L, Burstein H, Peluso R W, Polisky B, et al: Artificial miRNAs mitigate shRNA-mediated toxicity in the brain: implications for the therapeutic development of RNAi. Proc Natl Acad Sci USA 2008, 105:5868-5873.
23. Watakabe A, Ohtsuka M, Kinoshita M, Takaji M, Isa K, Mizukami H, Ozawa K, Isa T, Yamamori T: Comparative analyses of adeno-associated viral vector serotypes 1, 2, 5, 8 and 9 in marmoset, mouse and macaque cerebral cortex. Neurosci Res 2015, 93:144-157.
24. Levite M: Dopamine and T cells: dopamine receptors and potent effects on T cells, dopamine production in T cells, and abnormalities in the dopaminergic system in T cells in autoimmune, neurological and psychiatric diseases. Acta Physiol (Oxf) 2016, 216:42-89.
25. Ding S W, Voinnet O: Antiviral immunity directed by small RNAs. Cell 2007, 130:413-426.
26. Baek S T, Kerjan G, Bielas S L, Lee J E, Fenstermaker A G, Novarino G, Gleeson J G: Off-target effect of doublecortin family shRNA on neuronal migration associated with endogenous microRNA dysregulation. Neuron 2014, 82:1255-1262.
27. Pachner A R: Experimental models of multiple sclerosis. Curr Opin Neurol 2011, 24:291-299.
28. Procaccini C, De Rosa V, Pucino V, Formisano L, Matarese G: Animal models of Multiple Sclerosis. Eur J Pharmacol 2015, 759:182-191.
29. Rahim A A, Wong A M, Ahmadi S, Hoefer K, Buckley S M, Hughes D A, Nathwani A N, Baker A H, McVey J H, Cooper J D, Waddington S N: In utero administration of Ad5 and AAV pseudotypes to the fetal brain leads to efficient, widespread and long-term gene expression. Gene Ther 2012, 19:936-946.
30. Leonova K I, Brodsky L, Lipchick B, Pal M, Novototskaya L, Chenchik A A, Sen G C, Komarova E A, Gudkov A V: p53 cooperates with DNA methylation and a suicidal interferon response to maintain epigenetic silencing of repeats and noncoding RNAs. Proc Natl Acad Sci USA 2013, 110:E89-98.
31. Chiappinelli K B, Strissel P L, Desrichard A, Li H, Henke C, Akman B, Hein A, Rote N S, Cope L M, Snyder A, et al: Inhibiting DNA Methylation Causes an Interferon Response in Cancer via dsRNA Including Endogenous Retroviruses. Cell 2015, 162:974-986.
32. Roulois D, Loo Yau H, Singhania R, Wang Y, Danesh A, Shen S Y, Han H, Liang G, Jones P A, Pugh T J, et al: DNA-Demethylating Agents Target Colorectal Cancer Cells by Inducing Viral Mimicry by Endogenous Transcripts. Cell 2015, 162:961-973.
33. Pachner A R, Brady J, and Narayan K, Antibody-secreting cells in the central nervous system in an animal model of M S: phenotype, association with disability, and in vitro production of antibody. Journal of Neuroimmunology 2007, 190: 112-120.
34. Weber M S, Hemmer B and Cepok S, The role of antibodies in multiple sclerosis. Biochimica et Biophysica Acta-Molecular Basis of Disease 2011, 1812: 239-245.
35. Grimm D, Streetz K L, Kopling C L, Storm T A, Pandey K, Davis C R, Marion P, Salazar F, Kay M A, Fatality in mice due to oversaturation of cellular microRNA/short hairpin RNA pathways, Nature 2006, 441: 537-541.
36. Kariko K, Bhuyan P, Capodici J, Weissman D, Small interfering RNAs mediate sequence-independent gene suppression and induce immune activation by signaling through toll-like receptor 3, Journal of Immunology 2004, 172: 6545-6549.
37. Liu L, Botos I, Wang Y, Leonard J N, Shiloach J, Segal D M, Davies D R, Structural basis of toll-like receptor 3 signaling with double-stranded RNA, Science 2008, 320: 379-381.
38. Lemaire P A, Anderson E, Lary J, Cole J L, Mechanism of PKR activation by dsRNA, Journal of Molecular Biology 2008, 381: 351-360.
39. Mayo C B, Wong C J, Lopez P E, Lary J W, Cole J L, Activation of PKR by short stem-loop RNAs containing single-stranded arms, RNA 2016, 22: 1065-1075.
40. Puthenveetil S, Whitby L, Ren J, Kelnar K, Krebs J F, Beal pA, Controlling activation of the RNA-dependent protein kinase by siRNAs using site-specific chemical modification, Nucleic Acids Research 2006, 35: 4900-4911.
41. Barger S W, Goodwin M E, Porter M M, Beggs M L, Glutamate release from activated microglia requires the oxidative burst and lipid peroxidation, Journal of Neurochemistry 2007, 101: 1205-1213
42. McDonald J W, Athomsons S P, Hyrc K L, Choi Dw, Goldberg M P, Oligodendrocytes from forebrain are highly vulnerable to AMPA/kainate receptor-mediated excitotoxicity Nature Medicine 1998, 4: 291-297.
43. Leonova K I, Brodsky L, Lipchick B, Pal M, Novotoskaya L, Chenchik A A, Sen G C, Komarova E A, Gudkov A V, p53 cooperates with DNA methylation and a suicidal interferon response to maintain genetic silencing of repeats and noncoding RNAs, PNAS 2014, 110: E89-E98.
44. Roulois D, Loo Yau H, Singhania R, Wang Y, Danesh A, Shen S Y, Han H, Liang G, Jones P A, Pugh T J, O'Brien C, De Carvalho D D, DNA-demethylating agents targeting colorectal cancer cells by inducing viral mimicry by endogenous transcripts, Cell 2015, 162: 961-973.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing an animal model for a neuroinflammatory disease, the method comprising:
introducing into the striatum of an animal a composition comprising an inhibitory nucleic acid.

2. A method of producing an animal model for a neuroinflammatory disease, the method comprising:
introducing into the hippocampus of an animal a composition comprising an inhibitory nucleic acid.

3. A method of inducing neuroinflammation, microglial activation, white matter degeneration and injuries of large caliber axons in a rodent, the method comprising:
introducing into the striatum of a rodent a composition comprising an inhibitory nucleic acid.

4. The method of claim 1, wherein the inhibitory nucleic acid is further comprised within an adeno-associated virus (AAV) vector.

5. The method of claim 4, wherein the AAV vector is a recombinant self-complementary adeno-associated virus 8 (scAAV8) vector.

6. The method of claim 1, wherein the inhibitory nucleic acid is a short hairpin RNA (shRNA).

7. The method of claim 6, wherein the shRNA is about 20 to 30 base pairs in length.

8. The method of claim 6, wherein the shRNA comprises a nucleotide sequence that is at least 80% identical to a nucleotide sequence of a dopamine D1 receptor (Drd1) gene.

9. The method of claim 6, wherein the shRNA comprises a nucleotide sequence that is at least 80% identical to SEQ ID NO: 1, 3, 5, 7, or 13.

10. (canceled)

11. (canceled)

12. The method of claim 1, wherein the composition is introduced into a neuronal cell and/or a glial cell.

13. The method of claim 12, wherein the glial cell is a microglial cell, an astrocyte, and/or an oligodendrocyte.

14. The method of claim 1, wherein the composition is introduced by stereotaxic delivery.

15. The method of claim 1, wherein introducing results in over-proliferation and hypertrophy of microglial cells in the striatum.

16. The method of claim 1, wherein the neuroinflammatory disease is selected from the group consisting of: traumatic brain injury, progressive multifocal leukoencephalopathy, Parkinson's disease and multiple sclerosis.

17. The method of claim 1, wherein the neuroinflammatory disease is selected from multiple sclerosis and Alzheimer's disease.

18. (canceled)

19. The method of claim 1, wherein the animal is a rodent.

20.-30. (canceled)

31. A method of treating a neuroinflammatory disease, the method comprising:
administering a therapeutic agent to a non-human transgenic animal overexpressing a short hairpin RNA in the brain, wherein the non-human transgenic animal has a neuroinflammatory disease, to thereby treat the neuroinflammatory disease.

32. The method of claim 31, wherein the neuorinflammatory disease is selected from the group consisting of: traumatic brain injury, Alzheimer's disease, Parkinson's disease, progressive multifocal leukoencephalopathy, and multiple sclerosis.

33. The method of claim 31, wherein the short hairpin RNA comprises a nucleotide sequence that is at least 80% identical to SEQ ID NO: 1, 3, 5, or 13.

34. (canceled)

35. The method of claim 31, wherein treating results in delaying disease progression, reducing neuroinflammation, reducing white matter degeneration, reducing neurodegeneration, or any combination thereof.

36.-44. (canceled)


SEQUENCE LISTING (13 sequences)

SEQ ID NO: 1 - DNA, 57 bp, Artificial Sequence
  Short hairpin RNA
atacccttgtctgtgccgctcttcctgtcaagcggcacagacaagggtatttttttt

SEQ ID NO: 2 - DNA, 58 bp, Artificial Sequence
  Short hairpin RNA
aaaaaaaatacccttgtctgtgccgcttgacaggaagagcggcacagacaagggtatt

SEQ ID NO: 3 - DNA, 63 bp, Artificial Sequence
  Short hairpin RNA
ggcccttggatggcaattttactcttcctgtcaagtaaaattgccatccaagggccttttttt

SEQ ID NO: 4 - DNA, 73 bp, Artificial Sequence
  Short hairpin RNA
aaaaaaaggcccttggatggcaaatttactactgtccttctgacaggaagagtaaaattgccatccaagggcc

SEQ ID NO: 5 - DNA, 63 bp, Artificial Sequence
  Short hairpin RNA
ccctagggcttgctgttaagaaacttcctgtcatttcttaacagcaagccctagggttttttt

SEQ ID NO: 6 - DNA, 62 bp, Artificial Sequence
  Short hairpin RNA
aaaaaaaccctagggcttgctgttaagaaatacaggaagtttcttaacagcaagccctaggg

SEQ ID NO: 7 - DNA, 3497 bp, Mus musculus
tggagggactcgttgagtccaggggttttgggagaagtgactctaaagcaagggcatttggagagatgtggcaccaggggcttgaaggagaaatgcatgtattttaggctgtgtctcagaagaagaggcagcatccccgaaaagtgactgagattgaccaggaagaggccccagactcttagagtatgcttaaaagcctatgcattccttggggaatctagggaccagcggatgccagggaacagcgacctggagcactgaacccagaagacaggtggaaagcaagcggctcttcttcctggtatggcttggattgctatggagatgctcctgatggaacaccattgtgcttttgtccagacagcaactggggctggagaaggggctgggtggtgagtgattgggggaagtctggctaagcctggccaagaacgtgagggctaagccaccggaagtgctttccttctggaagatggctcctaacacttctaccatggatgagactggcctgccagtggagagggacttctcctttcgcatcctcacagcctgttttctgtccctgcttatcctgtccactctcttagggaatacccttgtctgtgccgctgtcatcaggtttcgacacctgcggtccaaggtgaccaacttctttgtcatctctttagctgtgtcagatctcttggtggctgtcttggtcatgccctggaaagctgtggctgagattgctggcttttggccctttgggtccttttgtaacatttgggtagcctttgacatcatgtgctccacggcatccatccttaacctctgtgtgatcagcgtggacaggtattgggctatctccagccctttccagtatgagaggaagatgactccgaaggcagccttcatcctgattagcgtagcatggactttgtctgttctcatatccttcattccagtgcagctaagctggcacaaggcaaaacctacatggcccttggatggcaattttacttccctggaagatgccgaggatgacaactgtgacacgaggttgagcaggacatacgccatttcatcctccctcataagcttttacatccccgtagccattatgatcgtcacttacaccagtatctacaggattgcccagaagcaaatccggcgcatctcagctttggagagggcagcagtccatgccaagaactgccagaccaccacaggtaatggaaaccctgtcgaatgctctcaatcagaaagttcctttaagatgtcctttaagagggagactaaagtcctgaagacactgtctgtgatcatgggggtattcgtgtgctgctggctccctttcttcatttcgaactgtatggtgcccttctgtggctctgaggagacccagccattctgcattgattccatcaccttcgatgtatttgtgtggtttggctgggcgaattcctccctgaaccccattatttatgcttttaatgccgattttcagaaggcattctcgaccctcttaggatgctatagactctgccctacaacgaataatgccatagagactgtaagcatcaacaacaacggggctgtgatgttttccagccaccatgagccccgaggctccatctccaaggactgtaatctggtttacctgatccctcatgctgtgggctcctctgaggacctgaagagggaggaggccggtggcatacctaagccactggagaagctgtccccggccttatcggtcatattggactatgacaccgatgtctctctagaaaagatccaacccgttacccacagcggacagcattcaacctgaatattgggttctcatctctgaagctatgagttccctagggcttgctgttaagaaattaacacgagacccctctgctgcttttggacagttacagaacttctccaactcactgactccagtgtattctctagccacaagggaaatgacttaggctctgaaatcctttttgaagcattatcttaggacactctatatatatatatttaaaaaaataatagggccaaaagtcaactgtaaacagcttcacttaaaaatcaaactttccaggaaggaagtgtgagagttgtttgctgtatccaaacaggtgctaaaactgtccaagcagttttcagattgtaaaggtaggtgcatgcctttgttaattaacttctctgacaataattgagccttacagcaagagtgggatttcttttttttttttctttctcagaattgacagatgcattgttgataatggttttatttatttatttatttatttatttatttatttattgtattatatggatattttaaatttatcatagtgaacctatatttagcttatttaatagaccgaaccaatgttaaatcagagactgaactgtccattgtactagcacttcatgagccaatgaaacagtacacatagactctgagattctgagctgggaatgacttctgggaaggcagcaaaggctgatgtggtggttccttaactcaacacagacacaaataaacacaaggtaagaagtgactaacactaccaatgctctgcccctaaaaatattttggaaagatatgttttttaaaaaagaagctactattgtgttctgaatgttttaaatggcagaggctttccccgaggcaaatttgcacttctgtaaatatctctgtaaaagccagctcaagaagaaatccaactttagatttctgtctgaatgtcgaggaagagcatatgccactttgtatttatgtaaaataattggccctccctgtcatttctcatttcatgcttgaaatggctttctgaaacaaacaaatgactgtccaggattaagatgtgcatcgaggtgaatgagctgtaaagtcacaggtcacagcagcccctccgatagttgggctcatcgctggttcttcatctgcccaggtctaaccaagccggctgctcaaggggctacttttgtagtgctttaatccgaatttagtatcctctcttaaaaaaaaaaaaaaaagctctttaatgttagtggtaaactagctaatgagctgtgcctcatcgtggaagtatacacttctgttgttggtggggggaatagaagaaccccttcccggtgtgtgttgagcatcacaaagccatcagcatcactagtcacaaatgctactttttttcctccctgtggttttgaatctagtttccaagtcatagcctggactgcaaaaagtaccatcaagtcccctcgggtgggggtattgcaacagtgtttcttttgttgtttgaaatgtttacaaggtgttctttggaagcagctgcaacatgtggatagaactgaaggaagggctgactggcttgctaacagtatctcctgcaggggtctgtgttacgtactttgaatgttttctcagctctaccgcttgtatgctttcttacataaaataaaattattttgtgaattcaaatcaaccccaaaaaaaaaaaaaaaaa

SEQ ID NO: 8 - DNA, 3543 bp, Mus musculus
gcagaagcgccttggcgggcggagcgcggagagcagagcgagcggcacaggagagggcgcagggttggcggagtcggggagcgtggtctcccagatcgggcatttggagagatgtggcaccaggggcttgaaggagaaatgcatgtattttaggctgtgtctcagaagaagaggcagcatccccgaaaagtgactgagattgaccaggaagaggccccagactcttagagtatgcttaaaagcctatgcattccttggggaatctagggaccagcggatgccagggaacagcgacctggagcactgaacccagaagacaggtggaaagcaagcggctcttcttcctggtatggcttggattgctatggagatgctcctgatggaacaccattgtgcttttgtccagacagcaactggggctggagaaggggctgggtggtgagtgattgggggaagtctggctaagcctggccaagaacgtgagggctaagccaccggaagtgctttccttctggaagatggctcctaacacttctaccatggatgagactggcctgccagtggagagggacttctcctttcgcatcctcacagcctgttttctgtccctgcttatcctgtccactctcttagggaatacccttgtctgtgccgctgtcatcaggtttcgacacctgcggtccaaggtgaccaacttctttgtcatctctttagctgtgtcagatctcttggtggctgtcttggtcatgccctggaaagctgtggctgagattgctggcttttggccctttgggtccttttgtaacatttgggtagcctttgacatcatgtgctccacggcatccatccttaacctctgtgtgatcagcgtggacaggtattgggctatctccagccctttccagtatgagaggaagatgactccgaaggcagccttcatcctgattagcgtagcatggactttgtctgttctcatatccttcattccagtgcagctaagctggcacaaggcaaaacctacatggcccttggatggcaattttacttccctggaagatgccgaggatgacaactgtgacacgaggttgagcaggacatacgccatttcatcctccctcataagcttttacatccccgtagccattatgatcgtcacttacaccagtatctacaggattgcccagaagcaaatccggcgcatctcagctttggagagggcagcagtccatgccaagaactgccagaccaccacaggtaatggaaaccctgtcgaatgctctcaatcagaaagttcctttaagatgtcctttaagagggagactaaagtcctgaagacactgtctgtgatcatgggggtattcgtgtgctgctggctccctttcttcatttcgaactgtatggtgcccttctgtggctctgaggagacccagccattctgcattgattccatcaccttcgatgtatttgtgtggtttggctgggcgaattcctccctgaaccccattatttatgcttttaatgccgattttcagaaggcattctcgaccctcttaggatgctatagactctgccctacaacgaataatgccatagagactgtaagcatcaacaacaacggggctgtgatgttttccagccaccatgagccccgaggctccatctccaaggactgtaatctggtttacctgatccctcatgctgtgggctcctctgaggacctgaagagggaggaggccggtggcatacctaagccactggagaagctgtccccggccttatcggtcatattggactatgacaccgatgtctctctagaaaagatccaacccgttacccacagcggacagcattcaacctgaatattgggttctcatctctgaagctatgagttccctagggcttgctgttaagaaattaacacgagacccctctgctgcttttggacagttacagaacttctccaactcactgactccagtgtattctctagccacaagggaaatgacttaggctctgaaatcctttttgaagcattatcttaggacactctatatatatatatttaaaaaaataatagggccaaaagtcaactgtaaacagcttcacttaaaaatcaaactttccaggaaggaagtgtgagagttgtttgctgtatccaaacaggtgctaaaactgtccaagcagttttcagattgtaaaggtaggtgcatgcctttgttaattaacttctctgacaataattgagccttacagcaagagtgggatttcttttttttttttctttctcagaattgacagatgcattgttgataatggttttatttatttatttatttatttatttatttatttattgtattatatggatattttaaatttatcatagtgaacctatatttagcttatttaatagaccgaaccaatgttaaatcagagactgaactgtccattgtactagcacttcatgagccaatgaaacagtacacatagactctgagattctgagctgggaatgacttctgggaaggcagcaaaggctgatgtggtggttccttaactcaacacagacacaaataaacacaaggtaagaagtgactaacactaccaatgctctgcccctaaaaatattttggaaagatatgttttttaaaaaagaagctactattgtgttctgaatgttttaaatggcagaggctttccccgaggcaaatttgcacttctgtaaatatctctgtaaaagccagctcaagaagaaatccaactttagatttctgtctgaatgtcgaggaagagcatatgccactttgtatttatgtaaaataattggccctccctgtcatttctcatttcatgcttgaaatggctttctgaaacaaacaaatgactgtccaggattaagatgtgcatcgaggtgaatgagctgtaaagtcacaggtcacagcagcccctccgatagttgggctcatcgctggttcttcatctgcccaggtctaaccaagccggctgctcaaggggctacttttgtagtgctttaatccgaatttagtatcctctcttaaaaaaaaaaaaaaaagctctttaatgttagtggtaaactagctaatgagctgtgcctcatcgtggaagtatacacttctgttgttggtggggggaatagaagaaccccttcccggtgtgtgttgagcatcacaaagccatcagcatcactagtcacaaatgctactttttttcctccctgtggttttgaatctagtttccaagtcatagcctggactgcaaaaagtaccatcaagtcccctcgggtgggggtattgcaacagtgtttcttttgttgtttgaaatgtttacaaggtgttctttggaagcagctgcaacatgtggatagaactgaaggaagggctgactggcttgctaacagtatctcctgcaggggtctgtgttacgtactttgaatgttttctcagctctaccgcttgtatgctttcttacataaaataaaattattttgtgaattcaaatcaaccccaaaaaaaaaaaaaaaaa

SEQ ID NO: 9 - DNA, 23 bp, Mus musculus
ggcccttggatggcaattttact

SEQ ID NO: 10 - DNA, 23 bp, Mus musculus
aagagcatatgccactttgtatt

SEQ ID NO: 11 - DNA, 10 bp, Artificial Sequence
  Loop sequence
cttcctgtca

SEQ ID NO: 12 - DNA, 48 bp, Artificial Sequence
  Random sequence
cctaaggttaagtcgccctcgctcgagcgagggcgacttaaccttagg

SEQ ID NO: 13 - DNA, 52 bp, Artificial Sequence
  Random shRNA
aacgtgtgaagctttaactaacttcctgtcattagttaaagcttcacacgtt